SEQUENCE LISTING (17 sequences)

SEQ ID NO: 1 - RNA, 38 nt, Artificial Sequence
  Synthetic Oligomer
guccuaagguagcgaaauuccuugucggguaaguuccg

SEQ ID NO: 2 - RNA, 42 nt, Artificial Sequence
  Synthetic Oligomer
gacucaacacggggaaacucaccagguccagacacaauaagg

SEQ ID NO: 3 - RNA, 24 nt, Artificial Sequence
  Synthetic Oligomer
uaaccguucucaucgcucuacgga

SEQ ID NO: 4 - RNA, 23 nt, Artificial Sequence
  Synthetic Oligomer
ucccccgcuacccgguacguucc

SEQ ID NO: 5 - RNA, 16 nt, Artificial Sequence
  Synthetic Oligomer
cgacaaggaauuucgc

SEQ ID NO: 6 - DNA, 22 bp, Artificial Sequence
  Synthetic Oligomer
gtctggacctggtgagtttccc

SEQ ID NO: 7 - RNA, 14 nt, Artificial Sequence
  Synthetic Oligomer
agagcgaugagaac

SEQ ID NO: 8 - RNA, 13 nt, Artificial Sequence
  Synthetic Oligomer
guaccggguagcg

SEQ ID NO: 9 - DNA, 32 bp, Artificial Sequence
  Synthetic Oligomer
ggctcctgcgacaaggaauuucgccaggagcc

SEQ ID NO: 10 - DNA, 32 bp, Artificial Sequence
  Synthetic Oligomer
ccgaggaccgacaaggaauuucgcgtcctcgg

SEQ ID NO: 11 - DNA, 32 bp, Artificial Sequence
  Synthetic Oligomer
cccagcaccgacaaggaauuucgcgtgctggg

SEQ ID NO: 12 - DNA, 38 bp, Artificial Sequence
  Synthetic Oligomer
ggctcctggtctggacctggtgagtttccccaggagcc

SEQ ID NO: 13 - DNA, 38 bp, Artificial Sequence
  Synthetic Oligomer
ccgaggacgtctggacctggtgagtttcccgtcctcgg

SEQ ID NO: 14 - RNA, 24 nt, Artificial Sequence
  Synthetic Oligomer
ccgacagagcgaugagaacgucgg

SEQ ID NO: 15 - RNA, 23 nt, Artificial Sequence
  Synthetic Oligomer
ccgagguaccggguagcgcucgg

SEQ ID NO: 16 - RNA, 25 nt, Artificial Sequence
  Synthetic Oligomer
uaaccguucucaucgcucuacggac

SEQ ID NO: 17 - RNA, 24 nt, Artificial Sequence
  Synthetic Oligomer
ucccccgcuacccgguacguuccc

(19) United States
(12) Patent Application Publication
(10) Pub. No.: US 2012/0231459 A1
(43) Pub. Date: Sep. 13, 2012

(54) CHEMILUMINESCENT PROBES FOR MULTIPLEX MOLECULAR QUANTIFICATION AND USES THEREOF

(75) Inventors: Kenneth A. BROWNE, Poway, CA (US); Ian WEEKS, Cardiff (GB)

(73) Assignee: GEN-PROBE INCORPORATED, San Diego, CA (US)

(21) Appl. No.: 13/445,780

(22) Filed: Apr. 12, 2012

Related U.S. Application Data
(63) Continuation-in-part of application No. 13/417,092, filed on Mar. 9, 2012, now abandoned.
(60) Provisional application No. 61/451,027, filed on Mar. 9, 2011.

(51) Int. Cl. G01N 21/76; C12N 7/00; C07D 401/12; C07D 219/06; C07H 21/04; C08B 37/00; C07H 5/06; C12N 1/00; C07K 17/02

(52) U.S. Cl. 435/6.11; 436/501; 435/243; 435/235.1; 435/317.1; 546/104; 530/409; 530/300; 536/23.1; 536/123.1; 530/395; 536/18.7; 530/396; 546/103

(57) ABSTRACT

A novel method is disclosed for simultaneous detection and quantification of two or more nucleic acid targets, without need for amplification. The method depends on spectral-temporal resolution of chemiluminescence emitted from independent hybridization-induced chemiluminescent signal (HICS) probes. The utility of this method has been demonstrated by use of resolvable N-linked acridinium and 2,7-dimethoxyacridinium ester labeled probes in a homogeneous assay for sensitive and simultaneous independent quantification of several bacterial and fungal target sequences. Compositions and kits for practicing the method of the present invention are also disclosed.

### CROSS REFERENCE TO RELATED APPLICATIONS

This application is a continuation-in-part of application Ser. No. 13/417,092, filed Mar. 9, 2012, which claims the benefit under 35 U.S.C. §119(e) to provisional patent application Ser. No. 61/451,027 filed Mar. 9, 2011, both of which are hereby incorporated by reference.

### FIELD OF THE INVENTION

This invention generally relates to the field of molecular diagnostics. In particular, the invention relates to the detection and quantification of multiple nucleic acid targets in a sample using spectrally and/or temporally resolved hybridization induced chemiluminescent signal (HICS) probes.

### BACKGROUND OF THE INVENTION

The presence of important and potentially pathogenic viral, bacterial, fungal, eukaryotic, or other species in dietary, agricultural, pharmaceutical, forensic, clinical, and environmental specimens requires rapid and sensitive methods for both identification and quantification of such organisms. In this context, molecular detection techniques such as sequencing (Meller et al., Proc. Natl. Acad. Sci. U.S.A. 2000, 97:1079-84; Shendure et al., Science 2005, 309:1728-32; Ozsolak et al., Nature 2009, 461:814-18), microarrays (Southern, E. M., J. Mol. Biol. 1975, 98:503-17; Fulton et al., Clin. Chem. 1997, 43:1749-56; Gunderson et al., Nat. Genet. 2005, 37:549-54), and nucleic acid probes (Tyagi et al., Nat. Biotechnol. 1996, 14:303-08; Nelson et al., Biochemistry 1996, 35:8429-38; Gaylord et al., Proc. Natl. Acad. Sci. U.S.A. 2002, 99:10954-57; Storhoff et al., Nat. Biotechnol. 2004, 22:883-87) are becoming increasingly important. For rapid time-to-result, sensitivity and specificity, probes are especially useful, but in order for them to be effective tools, target-bound probes need to be distinguished from probes that are not bound to target. This may be accomplished by separating bound probes from unbound probes (heterogeneous methods, as commonly carried out with radioisotopically labeled probes), then querying the bound fraction for the detectable signal. However, such methods increase the complexity of an assay and also provide opportunities for contamination with probes and/or target analytes in the reaction vessel.

Methods that avoid undue handling such as separations are favored. For example, self-reporting nucleic acid probes labeled with interacting fluorophore and quenching moieties (e.g., molecular beacons (MB; Tyagi et al., Nat. Biotechnol. 1996, 14:303-08), hairpin inversion probes (Browne, K. A., J. Am. Chem. Soc. 2005, 127:1989-94), and molecular torches (U.S. Pat. No. 6,534,274)) are powerful tools for analytical or diagnostic detection of nucleic acid targets. In the case of MBs (FIG. 1A), fluorescence induced by photoexcitation is lower in the absence of target and increases as binding to complementary nucleic acids induces increased distance between fluorophore and quenching moieties. Excess unbound probe exhibits lower emission than bound probe, and so separation is unnecessary, supporting a homogeneous assay format. Furthermore, careful selection of narrow band excitation sources or filters coupled with appropriate emission filters advantageously supports the simultaneous discrimination of two or more differently labeled fluorescent probes and, hence, can indicate the presence or absence of multiple analyte nucleic acids in a single assay.

Despite mitigating instrumentation and procedural designs, organic fluorescent probes tend to have a high background relative to the emitted signal caused by using a light source to initiate light emission phenomena, incomplete quenching, and the intrinsic photofluorescence of many species present in biological samples, including nucleic acids themselves. This means that the relationship between luminescence intensity and target nucleic acid concentration is not linear where the background contribution to the overall signal is significant and, thus, limits signal-based detection typically to nanomolar concentrations. Since organisms of interest may be present in low abundance, fluorescent probe-based detection often requires high yield target amplification methods such as the polymerase chain reaction (PCR; Saiki et al., Science 1988, 239:487-91) or transcription-mediated amplification (TMA; Brentano et al., NONRADIOACTIVE ANALYSIS OF BIOMOLECULES; Kessler, C., Ed.; Springer-Verlag: New York, 2000; pp. 374-80); however, amplification necessarily increases the complexity, time, and expense of the assay. Although time-to-detectable result can be improved by so-called real-time-amplification procedures in which fluorescent probes are monitored during amplification, it would still be advantageous to avoid amplification altogether.

By definition, emissions from chemiluminescent probes are the result of one or more chemical reactions. Since their detection is by observation without stimulation (e.g., excitation light), such probes are necessarily not perturbed by the interrogation technique. Therefore, unlike fluorescent probes, emission responses from chemiluminescent probes benefit from much lower intrinsic background emission so that such probes can be extremely sensitive and also offer a linear relationship between the magnitude of luminescence and the quantity of target nucleic acid present in specimens at low concentrations.

Acridinium ester (AE) chemiluminescent nucleic acid probes have traditionally had the AE moiety linked through the C9 acridinium position (C-linked) via an aminoalkyl group to a linear oligonucleotide containing 18-30 nucleotide (nt) units. (Nelson et al., NONISOTOPIC PROBING, BLOTTING, AND SEQUENCING; Kricka, L. J., Ed.; Academic Press: San Diego, Calif., 1995; pp. 391-428). After the probe has hybridized to its complementary target, bound and unbound AE probes could be physically separated, allowing detection of signal from only bound AE probe (in a heterogeneous assay). Alternatively, bound and unbound AE probes have been differentiated by mild alkaline treatment in which the ester moiety of the unbound AE probe is selectively hydrolyzed, rendering these probes non-chemiluminescent, while the ester of the AE probe hybridized to complementary target is protected from hydrolysis by its interaction with the nucleic acid double strands. This is the basis of the homogeneous hybridization protection assay. (Nelson et al. 1995, supra).

AE probe detection is initiated chemically by addition of hydrogen peroxide (H₂O₂) followed by a concentrated base solution, which leads to highly luminescent emissions from the released, excited N-methylacridone (FIG. 2). The signals are typically detected with a very sensitive photomultiplier tube (PMT) that sums photons from across the entire N-methylacridone emission spectrum (while typically reported as 430 nm, these emissions actually range from about 400 to 540 nm). This leads to a detection limit of about 600 fM (about 10³- to 10⁴-fold more sensitive than typical fluorescent probes) and a quantitative dynamic range spanning about 4 orders of magnitude. (Nelson et al. 1995, supra). Such high sensitivity supports either direct detection of nucleic acid analytes or detection of amplified analytes after only limited amplification.

Recently, a new probe type, the hybridization-induced chemiluminescent signal (HICS) probe, has been developed. (U.S. Pat. No. 7,169,554; U.S. Pub. No. 2007/0166759 A1; Brown et al., Org. Biomol. Chem. 2009, 7:386-94). HICS probes comprise an AE moiety conjugated through the N10 acridinium position (N-linked) to a site near one terminus of a stem-loop oligonucleotide and a quenching moiety attached near the opposite terminus (FIG. 1B), therefore resembling fluorescent MBs (FIG. 1A). Similar to MBs, upon hybridization of the HICS probe loop region to a complementary target nucleic acid, the stem dissociates, distancing the chemiluminophore from the quencher and allowing light emission to be detected upon chemical initiation. Thus, HICS probes self-report upon binding complementary nucleic acids, and probes in excess of target do not require physical or chemical separation from hybridized probes, allowing use in a homogeneous assay.

Unlike with their fluorescent counterparts, however, since no methods are available for simultaneous and independent determination of the emissions from different probes, quantitative use of HICS probes has been limited to measurement of only one analyte in each assay. To overcome this limitation, several novel developments are required:

(i) synthesis of two or more different emitter moieties that can be independently detected, for example, as a result of differences in wavelengths or kinetics of emission; (ii) adaptation of a typical luminometer to allow it to differentiate the emissions from the different emitters; and (iii) development of a protocol for carrying out multiple analyte detection.

Here, we report development of a homogeneous assay system for simultaneous determination of the emissions from different chemiluminescent probes and show its utility by independently measuring amounts of a pan-fungal nucleic acid sequence and a pan-bacterial nucleic acid sequence in seawater simultaneously, in a single assay, without the need for amplification.

### SUMMARY OF THE INVENTION

In one aspect, the present invention provides a homogeneous assay for quantifying two or more target nucleic acid sequences in a sample, e.g., a biological or environmental sample. First, the sample is contacted with a first hybridization induced chemiluminescent signal (HICS) probe that includes a first chemiluminescent molecule, under conditions allowing hybridization of the first HICS probe to a first target nucleic acid sequence. Second, the sample is contacted with a second HICS probe that includes a second chemiluminescent molecule, under conditions allowing hybridization of the second probe to a second target nucleic acid sequence that is different from the first target sequence. It is understood that these two steps can be carried out either simultaneously or sequentially without affecting the assay's performance. Next, chemical means are used to trigger the first and second chemiluminescent molecules in order to induce chemiluminescence emissions. In this regard, it is strongly preferable that the first and second chemiluminescent molecules have sufficiently different emission profiles to allow spectral and/or temporal resolution of the chemiluminescence emissions. Further, the chemiluminescence emissions from the first and second chemiluminescent molecules are detected using an optical detector capable of measuring optical signals at more than one wavelength. Finally, the target nucleic acid sequences in the sample are quantified based on the chemiluminescent emissions from the first and second chemiluminescent molecules. The quantification may be performed using specific activity values for the first and second chemiluminescent molecules, or alternatively, by comparing the chemiluminescent emissions from the first and second chemiluminescent molecules with reference emission values from a control sample.

In order to provide adequate spectral resolution, it is advantageous if the emission maxima of the first and second chemiluminescent molecules are separated by at least 20 nm, preferably at least 30 nm, more preferably at least 40 nm, and most preferably at least 50 nm. In some embodiments, the chemiluminescence emissions from the first and second chemiluminescent molecules can be resolved even if the emission maxima are fairly close together, provided that the emissions have significantly different kinetic profiles, thereby permitting temporal resolution in addition to, or independent of, the spectral resolution.

In some embodiments, the first and second chemiluminescent molecules include different acridinium ester (AE) moieties conjugated an acridinium position other than C9 to a stem-loop oligonucleotide. In other embodiments, the AE moieties are conjugated through the N10 acridinium position (N-linked) to a stem-loop oligonucleotide. Preferably, the AE moieties are conjugated to a site proximal to a terminus of a stem-loop oligonucleotide, and a quenching moiety is attached proximal to the opposite terminus of the oligonucleotide. Alternatively, it is contemplated that the AE and quenching moieties may be conjugated to sites that are proximal to the stem-loop junction or at other positions on the stem of a stem-loop oligonucleotide. In preferred embodiments, the first chemiluminescent molecule includes an unsubstituted AE moiety, and the second chemiluminescent molecule includes a 2,7-substituted AE moiety. In particularly preferred embodiments, the unsubstituted AE moiety is a 9-(2,6-dibromophenoxycarbonyl)-10-(3-carbonylpropyl) acridinium salt (e.g., iodide), whereas the substituted AE moiety is preferably a 2,7-dimethoxy-9-(2,6-dibromophenoxycarbonyl)-10-(3-carbonylpropyl) acridinium salt (e.g., iodide).

As noted above, the primary object of the present invention is to provide an improved method for detecting and quantifying multiple nucleic acid targets in the same sample. The target nucleic acid sequences can be from a mammalian (e.g., human) organism, a bacterium, a fungus, a virus, or any other organism of interest. For example, simultaneous quantitative determination of multiple infectious organisms represents one attractive application of the present invention. In some embodiments, the target nucleic acid sequences are selected from the group consisting of a pan-bacterial target sequence, a pan-fungal target sequence, a Chlamydia trachomatis (C. trachomatis) target sequence and a Neisseria gonorrhoeae (N. gonorrhoeae) target sequence.

The pan-bacterial target nucleic acid sequence may include a fragment of Escherichia coli O157:H7 (E. coli) 23S rRNA corresponding to nucleotides 1921-1958 and consisting of SEQ ID NO:1, allowing for a DNA equivalent thereof. In some embodiments, a pan-bacterial probe may include a stem-loop oligonucleotide having a target complementary base sequence consisting of SEQ ID NO:5, allowing for a DNA equivalent thereof. In particularly preferred embodiments, the stem-loop oligonucleotide may include a base sequence consisting of SEQ ID NO:9, SEQ ID NO:10 or SEQ ID NO:11, allowing for DNA equivalents thereof.

The pan-fungal target nucleic acid sequence may include a fragment of Candida albicans (C. albicans) 18S rRNA corresponding to nucleotides 1174-1217 and consisting of SEQ ID NO:2, allowing for a DNA equivalent thereof. In some embodiments, a pan-fungal probe may include a stem-loop oligonucleotide having a target complementary sequence consisting of SEQ ID NO:6, allowing for a RNA equivalent thereof. In particularly preferred embodiments, the stem-loop oligonucleotide may include a base sequence consisting of SEQ ID NO:12 or SEQ ID NO:13, allowing for RNA equivalents thereof.

The C. trachomatis target nucleic acid sequence may include a 23S rRNA fragment consisting of SEQ ID NO:3, allowing for a DNA equivalent thereof. In some embodiments, a C. trachomatis probe may include a stem-loop oligonucleotide having a target complementary sequence consisting of SEQ ID NO:7, allowing for a DNA equivalent thereof. In particularly preferred embodiments, the stem-loop oligonucleotide may include a base sequence consisting of SEQ ID NO:14, allowing for a DNA equivalent thereof.

The N. gonorrhoeae target nucleic acid sequence may include a 16S rRNA fragment consisting of SEQ ID NO:4, allowing for a DNA equivalent thereof. In some embodiments, a N. gonorrhoeae probe may include a stem-loop oligonucleotide having a target complementary base sequence consisting of SEQ ID NO:8, allowing for a DNA equivalent thereof. In particularly preferred embodiments, the stem-loop oligonucleotide may include a base sequence consisting of SEQ ID NO:15, allowing for a DNA equivalent thereof.

In some embodiments, the present assay provides a detection range of up to about 3 log units, preferably from about 200 amol to about 200 fmol, from about 500 amol to about 200 fmol, from about 500 amol to about 50 fmol, or from about 500 amol to about 5 fmol. In some embodiments, the assay can be completed in approximately 60 min per sample or less. The assay is capable of detecting two, three, four, five, or more different target sequences.

In another aspect, the present invention provides a composition that includes a 2,7-dimethoxy-9-(2,6-dibromophenoxycarbonyl)-10-(3-carbonylpropyl)acridinium salt (e.g., iodide). Since the composition is contemplated primarily for use in chemiluminescent labeling, in preferred embodiments the 2,7-dimethoxy-9-(2,6-dibromophenoxycarbonyl)-10-(3-carbonylpropyl)acridinium salt will be directly or indirectly conjugated to a small organic biomolecule, a macromolecule, a viral particle, a sub-cellular component, or a cell. The macromolecule conjugated to the 2,7-dimethoxy-9-(2,6-dibromophenoxycarbonyl)-10-(3-carbonylpropyl) acridinium salt may be selected from the group consisting of a protein, a peptide, a nucleic acid, an oligonucleotide, a polysaccharide, an oligosaccharide, a glycoprotein, a glycosaminoglycan, a lectin, a lipoprotein, a lipopolysaccharide, a hormone, a toxin, a cytokine, and a combination thereof. Preferably, the macromolecule is an oligonucleotide, more preferably, a stem-loop oligonucleotide.

In a further aspect, the present invention provides a kit that includes a 9-(2,6-dibromophenoxycarbonyl)-10-(3-carbonylpropyl)acridinium salt and reagent means for triggering a chemiluminescence emission. In some embodiments, the kit is intended for detecting and/or quantifying a target nucleic acid sequence in a sample, and the kit includes a 9-(2,6-dibromophenoxycarbonyl)-10-(3-carbonylpropyl)acridinium salt directly or indirectly conjugated to a stem-loop oligonucleotide and reagent means for triggering a chemiluminescence emission. In other embodiments, the kit is intended for detecting and/or quantifying at least two target nucleic acid sequences in a sample. The kit contains at least two different probes (e.g., HICS probes) that include at least two different chemiluminescent molecules having sufficiently different emission profiles to allow spectral and/or temporal resolution of their chemiluminescence emissions.

Preferably, the kit contains at least two different probes that include at least two chemiluminescent molecules having different acridinium ester (AE) moieties conjugated through an acridinium position other than C9 to a stem-loop oligonucleotide. More preferably, the AE moieties are conjugated through the N10 acridinium position (N-linked) to a stem-loop oligonucleotide. Preferably, the AE moieties are conjugated to a site proximal to a terminus of a stem-loop oligonucleotide, and a quenching moiety is attached proximal to the opposite terminus of the oligonucleotide. Alternatively, it is contemplated that the AE and quenching moieties may be conjugated to sites that are proximal to the stem-loop junction or at other positions on the stem of a stem-loop oligonucleotide. In preferred embodiments, one of the chemiluminescent molecules includes an unsubstituted AE moiety, and the second chemiluminescent molecule includes a 2,7-substituted AE moiety. In particularly preferred embodiments, the unsubstituted AE moiety is a 9-(2,6-dibromophenoxycarbonyl)-10-(3-carbonylpropyl)acridinium salt (e.g., iodide), whereas the substituted AE moiety is a preferably 2,7-dimethoxy-9-(2,6-dibromophenoxycarbonyl)-10-(3-carbonylpropyl)acridinium salt (e.g., iodide). In addition to the labeling reagent, it is contemplated that the kits of the present invention may further include reagent means for triggering a chemiluminescence emission.

### BRIEF DESCRIPTION OF THE DRAWINGS

FIGS. 1A-1B compare interactions and excitations/emissions of self-quenching fluorescent (A) and chemiluminescent (B) hairpin probes (commonly referred to as MB and HICS probes, respectively) with complementary target nucleic acids. Solid lines between nucleic acid strands represent base pairing; F and Q represent fluorophore and quencher moieties, respectively; and AE represents an acridinium ester chemiluminescent moiety.

FIG. 2 illustrates the progression of an unsubstituted AE in the presence of hydroxide or a combination of hydroxide and hydrogen peroxide to chemiluminescent N-methylacridone product. Using an unsubstituted, N-methyl AE (I) as a minimally complex model compound to describe AE pathways, with the understanding that the AEs in the current work share these pathways, FIG. 2 outlines many of the transformations and equilibria anticipated en route towards and away from the light emitting species. Elevated pH from addition of Detect 2 reagent (see Examples) shifts the equilibrium of H₂O₂ from Detect 1 reagent towards its anion form (ionic strength-corrected pKₐ₁=11.9) (Evans et al., Trans. Faraday Soc. 1949, 45:224-30). This facilitates formation of a 9-hydroperoxy adduct (II) and, in turn, a 9-hydroperoxy adduct anion (III) and a 1,2-dioxetane (IV), thereby comprising the observed rate limiting reactions. Loss of a phenolate results in a high-energy 1,2-dioxetan-3-one (V) that rapidly eliminates CO₂ to form an excited N-methylacridone (VI*). When VI* relaxes to VI, it emits light (or heat, though not measured in the current studies). The intramolecular transformations from IV to VI are anticipated to be rapid. The increased pH afforded upon addition of Detect 2 solution also drives competing processes that are not detected by light emission (dark reactions). Like the hydroperoxy anion, hydroxide can react at the acridinium 9 position, in this case forming a pseudobase (VII). Product analysis of I indicated that after 60 min at pH 9 in the absence of H₂O₂, ca. 90% of the reaction was composed of VII (Kaltenbach et al., Mikrochim. Acta 1992, 108:205-19). Thus, as other reactions deplete I, a substantial reserve of VII can equilibrate back to I. The alkaline solution additionally supports formation of a hydrated ester intermediate (VIII) and its essentially irreversible hydrolysis to a 9-carboxylic acid (IX) and a phenolate. King et al. calculated the pseudo-first-order base hydrolysis rate constant (k_{HO}-[HO⁻]) for a model AE in which k_{HO}- was 220 M⁻¹ s⁻¹ (King et al., Anal. Chem. 2007, 79:4169-76). Using this constant as a reasonable approximation in our work at pH 9 ([HO⁻]=1e⁻⁵ M), k_{HO}-[HO⁻¹] corresponds to 0.0022 s⁻¹ (τ_{1/2}=315 s) for I→VII→IX and, therefore, ester hydrolysis is not expected to contribute substantially to the dark processes on the timescales of the light-emitting reactions for most of the AE probes currently used.

FIG. 3 compares chemical structures of several acridinium ester reagents. Compound 1 is 9-(4-(2-succinimidyloxycarbonylethyl)phenoxycarbonyl)-10-methyl-acridinium trifluoromethanesulfonate. Compound 2 is 2,7-dimethoxy-9-(4-(2-benzyloxycarbonylethyl)phenoxycarbonyl)-10-methylacridinium trifluoromethanesulfonate. Compound 3 is 9-(2,6-dibromophenoxycarbonyl)-10-(3-succinimidyloxycarbonylpropyl) acridinium iodide. Compound 4 is 2,7-dimethoxy-9-(2,6-dibromophenoxycarbonyl)-10-(3-succinimidyloxycarbonylpropyl)acridinium iodide.

FIG. 4 illustrates chemical synthesis of N-linked 2,7-dimethoxyAE N-hydroxysuccinimide (NHS) ester label (compound 4). Intermediate compound 5 is bis(4-methoxyphenyl)amine. Intermediate compound 6 is N-(4-methoxyphenyl)-5-methoxyisatin. Intermediate compound 7 is 2,7-dimethoxyacridine-9-carboxylic acid. Intermediate compound 8 is 2,6-dibromophenyl 2,7-dimethoxyacridine-9-carboxylate. As noted above, the resulting compound 4 is 2,7-dimethoxy-9-(2,6-dibromophenoxycarbonyl)-10-(3-succinimidyloxycarbonylpropyl)acridinium iodide. Reagents and conditions: (i) CuI, K₂CO₃, L-proline, DMSO, 48 h, 90° C.; (ii) (ClCO)₂, AlCl₃, HCl; (iii) aq. KOH, 72 h; (iv) HCl; (v) SOCl₂; (vi) 2,6-dibromophenol; (vii) succinimidyl 4-iodobutanoate, 3 h, 150° C.

FIGS. 5A-5D show examples of time-resolved spectrograms following chemical initiation in the presence of: (A) 50 pmol each of EcoB1932-1947(−)HICS18 probe and EcoB1921-1958(+) target sequence; (B) 50 pmol of EcoB1932-1947(−)HICS18 probe alone; (C) 300 pmol each of CalA1185-1206(−)HICS87 probe and CalA1174-1217(+) target sequence; and (D) 300 pmol of CalA1185-1206(−)HICS87 probe alone. The curves show the counts per second (cps) summed over the preceding 0.4 sec interval (A, B) or the preceding 5 sec interval (C, D).

FIGS. 6A-6B illustrate representative time courses of chemiluminescent emissions for: (A) 0.5 pmol EcoB1932-1947(−)HICS18 probe plus 1 pmol EcoB1921-1958(+) target sequence; and (B) 0.5 pmol CalA1185-1206(−)HICS87 probe plus 1 pmol CalA1174-1217(+) target sequence. Each point shows the cps summed over a 0.25 s interval. Insets are output from 0.5 pmol probes in the absence of target. Lines through data points are non-linear best fits to a linear combination of exponential functions (Eq. 2).

FIGS. 7A-7B demonstrate spectral-temporal deconvolution of chemiluminescent emissions due to EcoB1932-1947(−)HICS18 and CalA1185-1206(−)HICS87 from mixtures containing 200 fmol of both probes plus various amounts of target (calibration curves) in a 1:1 mixture of seawater and 2× hybridization reagent. Open symbols are for EcoB1921-1958(+) (◯) or CalA1174-1217(+) (□) alone, while closed symbols are for EcoB1921-1958(+) () or CalA1174-1217(+) (▪) plus 50 fmol of the other target. In FIG. 7A, each point shows the wavelength-resolved cps (<450 nm) summed over 5×t_{1/2} of EcoB1932-1947(−)HICS18 (0-12.0 s). In FIG. 7B, each point shows the wavelength-resolved cps (>550 nm) summed from the end of the first 5×t_{1/2} of EcoB1932-1947(−)HICS18 to the end of the first 5×t_{1/2} of CalA1185-1206(−)HICS87 (12.0 s-184 s). All conditions were performed in at least duplicate. Lines through background-subtracted data points are linear best fits. Dashed lines indicate the minimum of one target that can be distinguished in the presence of a high quantity (200 fmol) of the other target.

### DETAILED DESCRIPTION OF THE INVENTION

For clarity of disclosure, and not by way of limitation, the detailed description of the invention is divided into the subsections that follow.

### A. DEFINITIONS

Unless defined otherwise, all technical and scientific terms used herein have the same meaning as is commonly understood by one of ordinary skill in the art to which this invention belongs. All patents, applications, published applications and other publications referred to herein are incorporated by reference in their entireties. If a definition set forth in this section is contrary to or otherwise inconsistent with a definition set forth in the patents, applications, published applications and other publications that are herein incorporated by reference, the definition set forth in this section prevails over the definition that is incorporated herein by reference.

As used herein, “a” or “an” means “at least one” or “one or more.”

Approximating language, as used herein throughout the specification and claims, may be applied to modify any quantitative or qualitative representation that could permissibly vary without resulting in a change in the basic function to which it is related. Accordingly, a value modified by a term such as “about” is not to be limited to the precise value specified, and may include values that differ from the specified value.

As used herein, the term “sample” generally refers to anything which may contain the analyte for which an assay is desired, more specifically target nucleic acids. The sample may be a biological sample, such as a biological fluid or a biological tissue. Examples of biological fluids include blood, plasma, serum, saliva, serum, sputum, urine, cerebral spinal fluid, tears, mucus, amniotic fluid, semen, stool, or the like. Biological tissues are aggregate of cells, usually of a particular kind of together with their intercellular substance that form one of the structural materials of a human structure, including connective, epithelium, muscle and nerve tissues. Examples of biological tissues also include organs, tumors, lymph nodes, arteries and individual cells.

As used herein, the term “nucleic acid” refers to a polynucleotide compound, which includes oligonucleotides, comprising nucleosides or nucleoside analogs that have nitrogenous heterocyclic bases or base analogs, covalently linked by standard phosphodiester bonds or other linkages. Nucleic acids include RNA, DNA, chimeric DNA-RNA polymers or analogs thereof. In a nucleic acid, the backbone may be made up of a variety of linkages, including one or more of sugar-phosphodiester linkages, peptide-nucleic acid (PNA) linkages (PCT Pub No. WO 95/32305), phosphorothioate linkages, methylphosphonate linkages, or combinations thereof. Sugar moieties in a nucleic acid may be ribose, deoxyribose, or similar compounds with substitutions, e.g., 2′ methoxy and 2′ halide (e.g., 2′-F) substitutions. Nitrogenous bases may be conventional bases (A, G, C, T, U), analogs thereof (e.g., inosine; The Biochemistry of the Nucleic Acids 5-36, Adams et al., ed., 11th ed., 1992), derivatives of purine or pyrimidine bases (e.g., N4-methyl deoxygaunosine, deaza- or aza-purines, deaza- or aza-pyrimidines, pyrimidines or purines with altered or replacement substituent groups at any of a variety of chemical positions, e.g., 2-amino-6-methylaminopurine, O6-methylguanine, 4-thio-pyrimidines, 4-amino-pyrimidines, 4-dimethylhydrazine-pyrimidines, and O4-alkyl-pyrimidines, or pyrazolo-compounds, such as unsubstituted or 3-substituted pyrazolo[3,4-d]pyrimidine (e.g., U.S. Pat. Nos. 5,378,825, 6,949,367 and PCT Pub. No. WO 93/13121)). Nucleic acids may include “abasic” positions in which the backbone does not have a nitrogenous base at one or more locations (U.S. Pat. No. 5,585,481), e.g., one or more abasic positions may form a linker region that joins separate oligonucleotide sequences together. A nucleic acid may comprise only conventional sugars, bases, and linkages as found in conventional RNA and DNA, or may include conventional components and substitutions (e.g., conventional bases linked by a 2′ methoxy backbone, or a polymer containing a mixture of conventional bases and one or more analogs). The term includes “locked nucleic acids” (LNA), which contain one or more LNA nucleotide monomers with a bicyclic furanose unit locked in a RNA mimicking sugar conformation, which enhances hybridization affinity for complementary sequences in ssRNA, ssDNA, or dsDNA (Vester et al., 2004, Biochemistry 43(42):13233-41).

As used herein, the interchangeable term “oligonucleotide” refers to nucleic acid polymers generally made of less than 1,000 nucleotide (nt), including those in a size range having a lower limit of about 2 to 5 nt and an upper limit of about 500 to 900 nt. Preferred oligonucleotides are in a size range having a 5 to 15 nt lower limit and a 50 to 500 nt upper limit, and particularly preferred embodiments are in a size range having a 10 to 20 nt lower limit and a 25 to 150 nt upper limit. Preferred oligonucleotides are made synthetically by using any well-known in vitro chemical or enzymatic method, and may be purified after synthesis by using standard methods, e.g., high-performance liquid chromatography (HPLC). Representative oligonucleotides discussed herein include detection probe oligonucleotides, such as stem-loop oligonucleotides (e.g., oligonucleotides forming MB and HICS probes).

As used herein, the term “label” generally refers to a molecular moiety or compound that can be detected or lead to a detectable response, which may be joined directly or indirectly to a nucleic acid probe. Direct labeling may use bonds or interactions to link label and probe, which includes covalent bonds, non-covalent interactions (hydrogen bonds, hydrophobic and ionic interactions), or chelates or coordination complexes. Indirect labeling may use a bridging moiety or linker (e.g. antibody, oligonucleotide, or other compound), which is directly or indirectly labeled, which may amplify a signal. Preferred labels include a detectable chemiluminescent moiety. Preferred chemiluminescent labels include acridinium ester (“AE”) and derivatives thereof (U.S. Pat. Nos. 5,639,604, 5,656,207 and 5,658,737). Preferred labels are detectable in a homogeneous assay in which bound labeled probe in a mixture exhibits a detectable change compared to that of unbound labeled probe, e.g., stability or differential degradation, without requiring physical separation of bound from unbound forms (e.g., U.S. Pat. Nos. 5,283,174, 5,656,207 and 5,658,737). General methods of synthesizing labels, attaching labels to nucleic acids, and detecting labels are well known (e.g., Sambrook et al., Molecular Cloning, A Laboratory Manual, 2nd ed. (Cold Spring Harbor Laboratory Press, Cold Spring Harbor, N.Y., 1989), Chapter 10; U.S. Pat. Nos. 4,581,333, 5,283,174, 5,547,842, 5,656,207 and 5,658,737).

As used herein, the term “contacting” means bringing two or more components together. “Contacting” can be achieved by mixing all the components in a fluid or semi-fluid mixture. “Contacting” can also be achieved when one or more components are brought into physical contact with one or more other components on a solid surface such as a solid tissue section or a substrate.

As used herein, “detection” of the target nucleic acids generally relies on probes in which signal production is linked to the presence of the target sequence because a change in signal results only when the labeled probe binds to amplified product, such as in a molecular beacon (MB), molecular torch, hybridization switch, or hybridization-induced chemiluminescence signal (HICS) probe (e.g., U.S. Pat. Nos. 5,118,801, 5,312,728, 5,925,517, 6,150,097, 6,361,945, 6,534,274, 6,835,542, 6,849,412, 7,169,554 and 8,034,554; and U.S. Pub. Nos. 2006/0194240 A1 and 2007/0166759 A1). Preferred probes use a chemiluminescent label attached to one end of the probe and an interacting compound (e.g., quencher) attached to another location of the probe to inhibit signal production from the label when the probe is in a “closed” conformation that indicates it is not hybridized to its target sequence, but a detectable optical signal is produced when the probe is hybridized to the target sequence which changes its conformation to “open”. Detection of an optical signal from directly or indirectly labeled probes that specifically associate with the target sequence indicates the presence of the target nucleic acid.

Further, the terms “detecting,” “assessing” and “measuring” as used herein are intended to include both quantitative and qualitative determination in the sense of obtaining an absolute value for the amount or concentration of the analyte present in the reaction system, and also of obtaining an index, ratio, percentage, visual or other value indicative of the level of analyte in the reaction system. In contrast, the term “quantifying” specifically refers to quantitative determinations.

### B. MULTIPLEX QUANTITATIVE ASSAYS

As noted above, the first aspect of the invention relates to a homogeneous assay for quantifying two or more target nucleic acid sequences in a sample, e.g., a biological or environmental sample. First, the sample is contacted with a first hybridization induced chemiluminescent signal (HICS) probe that includes a first chemiluminescent molecule, under conditions allowing hybridization of the first HICS probe to a first target nucleic acid sequence. Second, the sample is contacted with a second HICS probe that includes a second chemiluminescent molecule, under conditions allowing hybridization of the second probe to a second target nucleic acid sequence that is different from the first target sequence. It is understood that these two steps can be carried out either simultaneously or sequentially without affecting the assay's performance. Next, chemical means are used to trigger the first and second chemiluminescent molecules in order to induce chemiluminescence emissions. In this regard, it is strongly preferable that the first and second chemiluminescent molecules have sufficiently different emission profiles to allow spectral and/or temporal resolution of the chemiluminescence emissions. Further, the chemiluminescence emissions from the first and second chemiluminescent molecules are detected using an optical detector capable of measuring optical signals at more than one wavelength. Finally, the chemiluminescent emissions from the first and second chemiluminescent molecules are compared with reference values, thereby quantifying the target nucleic acid sequences in the sample.

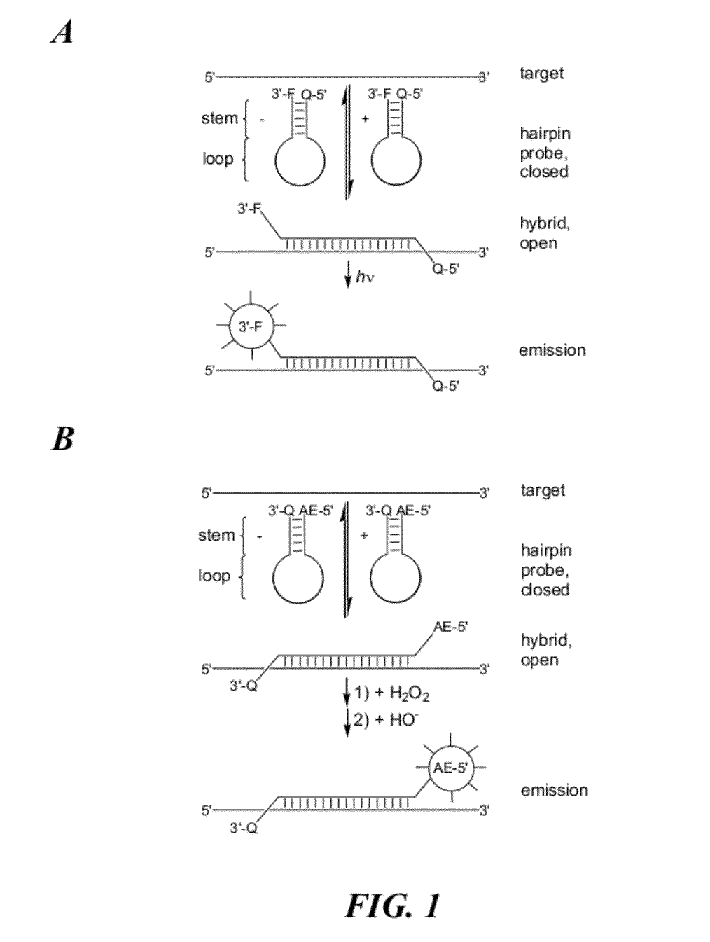

In order to allow detection of two nucleic acid analytes using pairs of HICS probes, the two probes must differ both in their ability to recognize different oligonucleotide sequences and in the nature of the luminescence they emit. Each probe molecule must incorporate at least four features: (i) an oligonucleotide strand complementary to an oligonucleotide sequence in a particular target nucleic acid marker; (ii) extensions on either side of the aforesaid oligonucleotide sequence by oligonucleotide strands (stem arms) complementary to each other so that they cause the two ends of the oligonucleotide to bind to each other in a hairpin-like arrangement; (iii) a chemiluminophore unit attached to one end of the oligonucleotide sequence via an aminoalkyl group or related spacer; (iv) a quencher for the luminescence emitted by the chemiluminophore, attached to the other end of the oligonucleotide sequence via an appropriate spacer (see FIG. 1). The stem arms may be partially or completely complementary to the target sequence. When the stem arms hybridize to themselves in the absence of target, the target complementary sequence typically forms a loop structure.

The starting point is to design an appropriate pair of reagents to introduce at least two differentiable chemiluminophores. As noted above, in order to provide adequate spectral resolution, it is desirable that the emission maxima of the chemiluminophores be separated by at least 20 nm, preferably at least 30 nm, more preferably at least 40 nm, and most preferably at least 50 nm. Alternatively, it is desirable that the chemiluminescence emissions from the chemiluminophores can be resolved even if the emission maxima are fairly close together, provided that the emissions have significantly different kinetic profiles, thereby permitting temporal resolution in addition to, or independent of, the spectral resolution.

In some embodiments of the present invention, the first and second chemiluminescent molecules include different acridinium ester (AE) moieties conjugated at an acridinium position other than C9 to a stem-loop oligonucleotide. In other embodiments, the AE moieties are conjugated through the N10 acridinium position (N-linked) to a stem-loop oligonucleotide. Preferably, the AE moieties are conjugated to a site proximal to a terminus of a stem-loop oligonucleotide, and a quenching moiety is attached proximal to the opposite terminus of the oligonucleotide. Alternatively, it is contemplated that the AE and quenching moieties may be conjugated to sites that are proximal to the stem-loop junction or at other positions on the stem of a stem-loop oligonucleotide. In preferred embodiments, the first chemiluminescent molecule includes an unsubstituted AE moiety, and the second chemiluminescent molecule includes a 2,7-substituted AE moiety. In some particularly preferred embodiments, the unsubstituted AE moiety is a 9-(2,6-dibromophenoxycarbonyl)-10-(3-carbonylpropyl)acridinium salt (e.g., iodide), whereas the substituted AE moiety is preferably a 2,7-dimethoxy-9-(2,6-dibromophenoxycarbonyl)-10-(3-carbonylpropyl) acridinium salt (e.g., iodide).

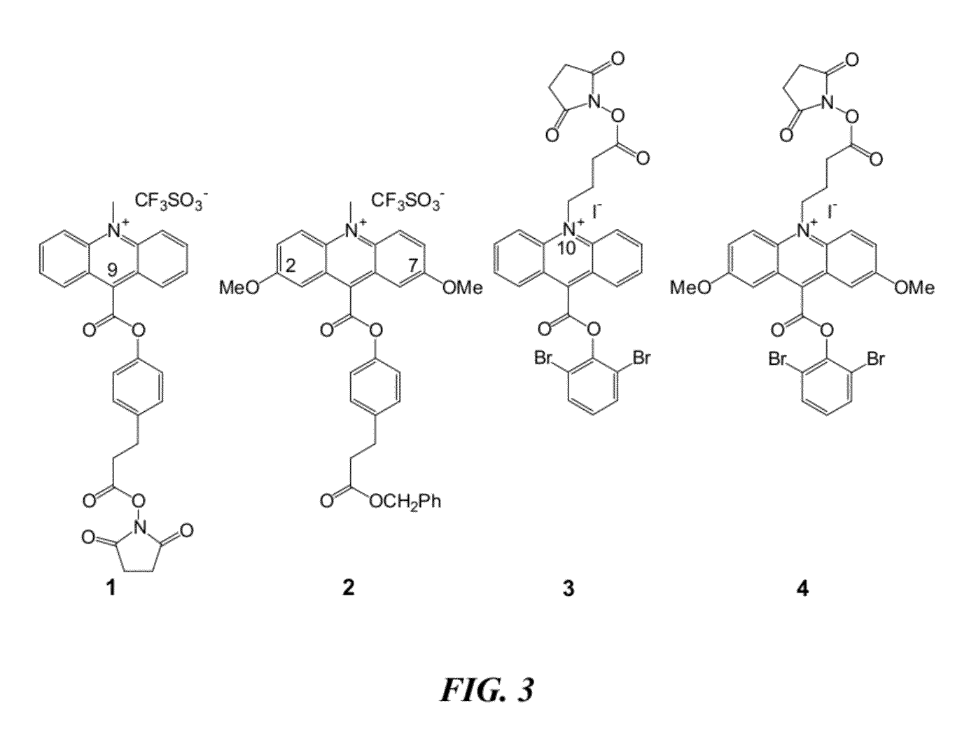

9-(4-(2-Succinimidyloxycarbonylethyl)phenoxycarbonyl)-10-methylacridinium trifluoromethanesulfonate (compound 1, FIG. 3) is an unsubstituted AE labeling reagent that has been used to generate biological probes having C-linked AE moieties. (U.S. Pat. No. 4,946,958). It includes (i) an active group (N-hydroxysuccinimide, NHS) capable of ready attachment to an amino substituent in the unlabeled probe, (ii) a leaving group (phenoxide) that is eliminated during treatment with alkaline H₂O₂, and (iii) a chemiluminescent group (the AE unit, which is converted into excited state N-methylacridone during treatment with alkaline H₂O₂). Substituents on the acridine unit have a major influence on the wavelength of emission. We investigated one design pathway, wavelength-resolvable AE probes, in our attempt to develop appropriate multiplex detection systems. This required a substituted AE that emitted luminescence distinct from that characteristic of compound 1.

While physicochemical rationalization can assist in the design of longer wavelength-emitting N-alkylacridone precursors, empirical studies are required to discover AE derivatives that balance the increased emission wavelength (e.g., ≧50 nm) needed to distinguish them from the unsubstituted AE in aqueous solution with stability in aqueous environments and the ability to initiate chemiluminescence under conditions that will not damage oligonucleotide hybrids. Woodhead et al. posited that extending the conjugation of the acridinium ring system to a 2,4-pentadienoic acid moiety would reduce the energy gap between the excited and ground state N-methylacridone species, resulting in a bathochromic shift of chemiluminescent emission wavelength by up to 80 nm compared to an unmodified AE. (U.S. Pat. No. 5,656,207). However, Law et al. later synthesized a closely related AE and found only a 37 nm increase in peak emission wavelength in an acetonitrile solution. (U.S. Pat. No. 5,879,894). Law et al. also extended their studies to include AEs fused to additional aromatic rings and reported that the excited N-methylbenzacridone from the angular benz[α]acridinium ester derivative increased peak emission by only 11 nm while the excited N-methylbenzacridone from the linear benz[b]AE derivative increased peak emission wavelength by 95 nm in acetonitrile solutions. (Id.) In a series of N-sulfopropyl AEs, Natrajan et al. reported that incorporation of a single methoxy group at the acridinium ring 3 position led to a slight (8 nm) hypsochromic shift in the maximum emission wavelength whereas incorporation of the substituent at the 2 or 4 position led to increases in the peak emission wavelength of 32 and 52 nm, respectively. (Natrajan et al., Anal. Biochem. 2010, 406:204-13; U.S. Pat. Nos. 7,309,615 and 7,785,904). Natrajan et al. also found that 2,7-dimethoxy substitution of N-sulfopropyl AE led to a 58 nm increase in the maximum emission wavelength but was accompanied by a significant decrease in stability in an aqueous solution compared to other substitutions. (Id.)

We have previously synthesized a considerable variety of AEs with different substituents on both the phenoxide and acridine rings. (See, e.g., Batmanghelich et al., J. Photochem. Photobiol., A 1991, 56:249-54; Batmanghelich et al., J. Photochem. Photobiol., B 1992, 12:193-201; Smith et al., J. Photochem. Photobiol., A 2000, 132:181-91; Smith et al., J. Photochem. Photobiol., A 2009, 203:72-79; Li, Z., Ph.D. Thesis, University of Wales, Swansea, U.K., 1998). In the course of those studies, we sometimes observed that, relative to an unmodified AE, the changes in peak chemiluminescent wavelengths on introduction of a particular substituent are not always the same as the changes reported in the literature for similar systems. Effects of different solvents used may account for at least part of the differences in observed photonic behaviors. However, we did observe that the emission wavelength of 2,7-dimethoxy-9-(4-benzyloxycarbonylethylphenoxycarbonyl)-10-methylacridiniumtrifluoromethanesulfonate (compound 2, FIG. 3) was 55 nm longer than that from compound 1 in aqueous solution, presumably because of electron donation (+I) from the two methoxy groups.

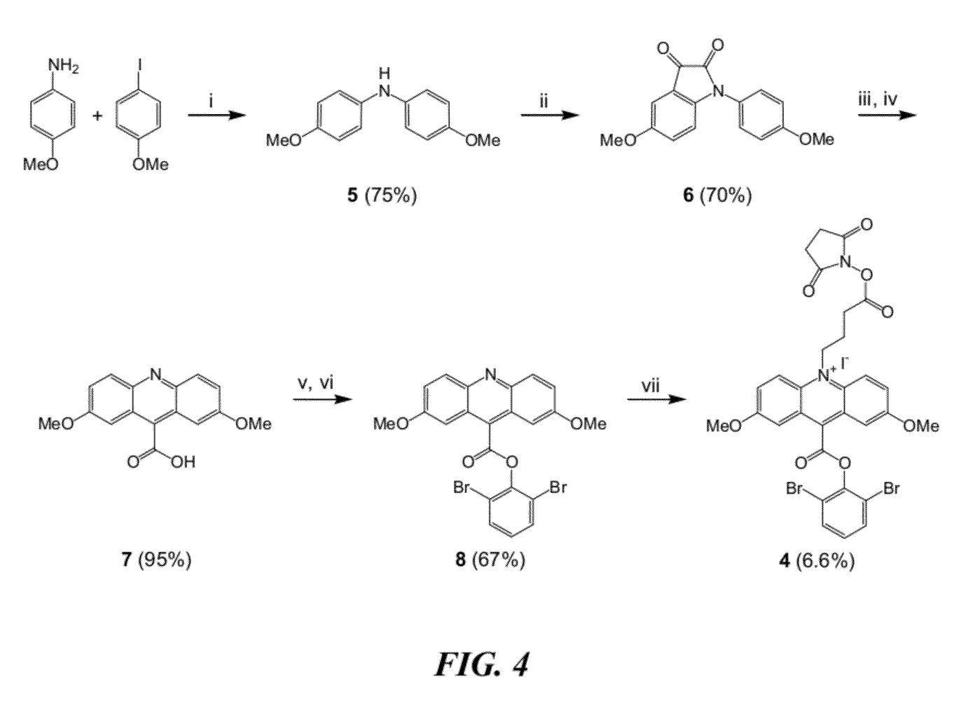

9-(2,6-Dibromophenoxycarbonyl)-10-(3-succinimidyloxycarbonylpropyl) acridinium iodide labeling reagent (compound 3, FIG. 3) was used to produce previously reported HICS probes. (Brown et al., Org. Biomol. Chem. 2009, 7:386-94). It incorporates two features that differ significantly from labeling compound 1. First, it results in N-linked attachment of the AE label to oligonucleotides rather than through the phenolic leaving group. When chemically initiated, light from these probes is emitted from an excited N-alkylacridone species that remains linked to the oligonucleotide. In the absence of target, the stem forces the emitter and the quencher to remain proximal and, hence, allows little energy to be emitted as detectable light upon initiation. Second, the bulky, electron-withdrawing (−I) bromo substituents help protect the ester from facile hydrolysis and also decrease the pKa of the leaving group phenol, thereby supporting formation of the excited N-alkylacridone under mildly alkaline conditions that maintain nucleic acid hybrids. We selected this compound for labeling our unsubstituted AE probes and the 2,7-dimethoxy analogue (compound 4, FIG. 3) for labeling the differentiable probes. The organic syntheses of labeling compound 4, as well as its intermediates, is shown in FIG. 4 and described in detail in Example 1.

As noted above, the primary object of the present invention is to provide an improved method for detecting and quantifying multiple nucleic acid targets in the same sample. The target nucleic acid sequences can be from a mammalian (e.g., human) organism, a bacterium, a fungus, a virus, or any other organism of interest. For example, simultaneous quantitative determination of multiple infectious organisms represents one attractive application of the present invention. The target nucleic acid sequences may be selected from the group consisting of a pan-bacterial target sequence, a pan-fungal target sequence, a Chlamydia trachomatis (C. trachomatis) target sequence and a Neisseria gonorrhoeae (N. gonorrhoeae) target sequence. Selected target and probe sequences are set forth in Table 1.

TABLE 1
Selected target and probe nucleic
acid sequences.

| SEQ | | |
|---|---|---|
| ID | Nucleotide Sequene (5′ → 3′) | Length |
| 1 | GUCCUAAGGU AGCGAAAUUC CUUGUCGGGU | 38 |
| | AAGUUCCG | |
| 2 | GACUCAACAC GGGGAAACUC ACCAGGUCCA | 42 |
| | GACACAAUAA GG | |
| 3 | UAACCGUUCU CAUCGCUCUA CGGA | 24 |
| 4 | UCCCCCGCUA CCCGGUACGU UCC | 23 |
| 5 | CGACAAGGAA UUUCGC | 16 |
| 6 | GTCTGGACCT GGTGAGTTTC CC | 22 |
| 7 | AGAGCGAUGA GAAC | 14 |
| 8 | GUACCGGGUA GCG | 13 |
| 9 | GGCTCCTGCG ACAAGGAAUU UCGCCAGGAG | 32 |
| | CC | |
| 10 | CCGAGGACCG ACAAGGAAUU UCGCGTCCTC | 32 |
| | GG | |
| 11 | CCCAGCACCG ACAAGGAAUU UCGCGTGCTG | 32 |
| | GG | |
| 12 | GGCTCCTGGT CTGGACCTGG TGAGTTTCCC | 38 |
| | CAGGAGCC | |
| 13 | CCGAGGACGT CTGGACCTGG TGAGTTTCCC | 38 |
| | GTCCTCGG | |
| 14 | CCGACAGAGC GAUGAGAACG UCGG | 24 |
| 15 | CCGAGGUACC GGGUAGCGCU CGG | 23 |
| 16 | UAACCGUUCU CAUCGCUCUA CGGAC | 25 |
| 17 | UCCCCCGCUA CCCGGUACGU UCCC | 24 |

The pan-bacterial target nucleic acid sequence may include a fragment of Escherichia coli O157:H7 (E. coli) 23S rRNA corresponding to nucleotides 1921-1958 and consisting of SEQ ID NO:1, allowing for a DNA equivalent thereof. In some embodiments, the pan-bacterial probe may include a stem-loop oligonucleotide having a target complementary base sequence consisting of SEQ ID NO:5, allowing for a DNA equivalent thereof. In particularly preferred embodiments, the stem-loop oligonucleotide may include a base sequence consisting of SEQ ID NO:9, SEQ ID NO:10 or SEQ ID NO:11, allowing for DNA equivalents thereof. In some embodiments, the probes may include one or more modified nucleotides such as 2′-O-methylribose.

The pan-fungal target nucleic acid sequence may include a fragment of Candida albicans (C. albicans) 18S rRNA corresponding to nucleotides 1174-1217 and consisting of SEQ ID NO:2, allowing for a DNA equivalent thereof. In some embodiments, the pan-fungal probe may include a stem-loop oligonucleotide having a target complementary sequence consisting of SEQ ID NO:6, allowing for a RNA equivalent thereof. In particularly preferred embodiments, the stem-loop oligonucleotide may include a base sequence consisting of SEQ ID NO:12 or SEQ ID NO:13, allowing for RNA equivalents thereof.

The C. trachomatis target nucleic acid sequence may include a 23S rRNA fragment consisting of SEQ ID NO:3, allowing for a DNA equivalent thereof. In some embodiments, the C. trachomatis probe may include a stem-loop oligonucleotide having a target complementary sequence consisting of SEQ ID NO:7, allowing for a DNA equivalent thereof. In particularly preferred embodiments, the stem-loop oligonucleotide may include a base sequence consisting of SEQ ID NO:14, allowing for a DNA equivalent thereof. In some embodiments, the probe may include one or more modified nucleotides such as 2′-β-methylribose.

The N. gonorrhoeae target nucleic acid sequence may include a 16S rRNA fragment consisting of SEQ ID NO:4, allowing for a DNA equivalent thereof. In some embodiments, the N. gonorrhoeae probe may include a stem-loop oligonucleotide having a target complementary base sequence consisting of SEQ ID NO:8, allowing for a DNA equivalent thereof. In particularly preferred embodiments, the stem-loop oligonucleotide may include a base sequence consisting of SEQ ID NO:15, allowing for a DNA equivalent thereof. In some embodiments, the probe may include one or more modified nucleotides such as 2′-O-methylribose.

As described in detail in Example 5, the assays of the present invention allow a rapid (about 60 min per sample), sensitive (about 500 amol to 200 fmol), and homogeneous detection and quantification (dynamic range spanning up to 3 log concentration units) process without enzymatic amplification of target nucleic acid sequences. Although the working examples provided herein refer to simultaneous detection and quantification of two target sequences, it is contemplated that the assay lends itself to providing further value by addition of extra detection channels to the luminometer and by including one or more additional spectrally and temporally resolved probes to other analytes or internal controls.

### C. COMPOSITIONS AND KITS FOR CHEMILUMINESCENT DETECTION

As noted above, the present invention also provides various compositions and kits for chemiluminescent detection. In one aspect, the invention provides a composition that includes a 2,7-dimethoxy-9-(2,6-dibromophenoxycarbonyl)-10-(3-carbonylpropyl) acridinium salt (e.g., iodide). Since the composition is contemplated primarily for use in chemiluminescent labeling, in preferred embodiments the 2,7-dimethoxy-9-(2,6-dibromophenoxycarbonyl)-10-(3-carbonylpropyl)acridinium salt will be directly or indirectly conjugated to a small organic biomolecule, a macromolecule, a viral particle, a sub-cellular component, or a cell. The macromolecule conjugated to the 2,7-dimethoxy-9-(2,6-dibromophenoxycarbonyl)-10-(3-carbonylpropyl)acridinium salt may be selected from the group consisting of a protein, a peptide, a nucleic acid, an oligonucleotide, a polysaccharide, an oligosaccharide, a glycoprotein, a glycosaminoglycan, a lectin, a lipoprotein, a lipopolysaccharide, a hormone, a toxin, a cytokine, and a combination thereof. Preferably, the macromolecule is an oligonucleotide, more preferably, a stem-loop oligonucleotide or any other oligonucleotide that can be employed in a molecular beacon (MB), molecular torch, hybridization switch, or hybridization-induced chemiluminescence signal (HICS) probe (e.g., U.S. Pat. Nos. 5,118,801, 5,312,728, 5,925,517, 6,150,097, 6,361,945, 6,534,274, 6,835,542, 6,849,412, 7,169,554 and 8,034,554; and U.S. Pub. Nos. 2006/0194240 A1 and 2007/0166759 A1).

In another aspect, the invention provides a kit that includes a 9-(2,6-dibromophenoxycarbonyl)-10-(3-carbonylpropyl)acridinium salt and reagent means for triggering a chemiluminescence emission. In some embodiments, the kit is intended for detecting and/or quantifying a target nucleic acid sequence in a sample, and the kit includes a 9-(2,6-dibromophenoxycarbonyl)-10-(3-carbonylpropyl)acridinium salt directly or indirectly conjugated to a stem-loop oligonucleotide and reagent means for triggering a chemiluminescence emission. In other embodiments, the kit is intended for detecting and/or quantifying at least two target nucleic acid sequences in a sample. The kit contains at least two different probes (e.g., HICS probes) that include at least two different chemiluminescent molecules having sufficiently different emission profiles to allow spectral and/or temporal resolution of their chemiluminescence emissions.

Preferably, the kit contains at least two different probes that include at least two chemiluminescent molecules having different acridinium ester (AE) moieties conjugated through an acridinium position other than C9 to a stem-loop oligonucleotide. More preferably, the AE moieties are conjugated through the N10 acridinium position (N-linked) to a stem-loop oligonucleotide. Preferably, the AE moieties are conjugated to a site proximal to a terminus of a stem-loop oligonucleotide, and a quenching moiety is attached proximal to the opposite terminus of the oligonucleotide. Alternatively, it is contemplated that the AE and quenching moieties may be conjugated to sites that are proximal to the stem-loop junction or at other positions on the stem of a stem-loop oligonucleotide. In preferred embodiments, one of the chemiluminescent molecules includes an unsubstituted AE moiety, and the second chemiluminescent molecule includes a 2,7-substituted AE moiety. In particularly preferred embodiments, the unsubstituted AE moiety is a 9-(2,6-dibromophenoxycarbonyl)-10-(3-carbonylpropyl)acridinium salt (e.g., iodide), whereas the substituted AE moiety is a preferably 2,7-dimethoxy-9-(2,6-dibromophenoxycarbonyl)-10-(3-carbonylpropyl)acridinium salt (e.g., iodide). In addition to the labeling reagent, it is contemplated that the kits of the present invention may further include reagent means for triggering a chemiluminescence emission.

The invention can be better understood by reference to following non-limiting examples.

### EXAMPLES

### Example 1

### Synthesis of Chemiluminescent Labeling Molecules

### 1(a) General

Chemicals and reagents were obtained from Aldrich Chemical Co. and were used without further purification unless otherwise stated. Tetrahydrofuran (THF) was distilled from sodium benzophenone ketyl. Other solvents were purified by standard procedures. (Vogel, A. I., VOGEL'S TEXTBOOK OF PRACTICAL ORGANIC CHEMISTRY, 5th ed.; Longman: Harlow, U.K., 1989; Perrin et al., PURIFICATION OF LABORATORY CHEMICALS, 3rd ed.; Pergamon Press: Oxford, U.K., 1988). Melting point determinations were performed by the open capillary method using a Gallenkamp melting point apparatus and are reported uncorrected. ¹H and ¹³C NMR spectra were recorded on an AV500 spectrometer operating at 500 MHz for ¹H or 125 MHz for ¹³C measurements. Chemical shifts δ are reported in parts per million (ppm) relative to tetramethylsilane, and coupling constants J are in Hz. Low- and high-resolution mass spectra were recorded on a GCT-Premier spectrometer, electron impact (EI) at 70 eV. Atmospheric pressure chemical ionization (APCI) mass spectra were acquired on a Waters LCT Premier XE instrument. Electrospray (ES) mass spectrometric analyses were performed on a ZQ4000 instrument in positive ionization mode. Column chromatography was carried out using Fisher Scientific silica 60A (35-70 μm).

### 1(b) Syntheses of Labeling Compounds

Compound 3 was obtained by a published procedure. (Brown et al., supra). The preparation of compound 4 followed a similar route but with additional steps to produce 2,7-dimethoxyacridine-9-carboxylic acid (7) prior to esterification and quaternization (FIG. 4). Bis(4-methoxyphenyl)amine (5) was prepared in 75% yield according to a standard literature procedure. (Zhang et al., J. Org. Chem. 2005, 70:5164-73). Reaction of compound 5 with oxalyl chloride in dichloromethane followed by treatment with aluminum chloride gave N-(4-methoxyphenyl)-5-methoxyisatin (6) in 70% yield, which on treatment with potassium hydroxide under reflux conditions for 72 h gave 2,7-dimethoxyacridine-9-carboxylic acid (7) in 95% yield. A mixture of compound 7 and freshly redistilled thionyl chloride was heated under reflux for 3 h to afford the corresponding acid chloride, which on reaction with 2,6-dibromophenol in pyridine at 70° C. gave 2,6-dibromophenyl 2,7-dimethoxyacridine-9-carboxylate (8) in 67% yield. Reaction of compound 8 with succinimidyl 4-iodobutanoate at 150° C. for 3 h gave 2,7-dimethoxy-9-(2,6-dibromophenoxycarbonyl)-10-(3-succinimidyloxycarbonylpropyl)acridinium iodide (4) in 6.6% yield. The low yield resulted from a very slow reaction, even at 150° C. Use of even more forcing conditions caused some decomposition of the starting material, whereas under the conditions reported most of the unreacted starting material could be recovered without detectable side products. Thus, all except the last step gave reasonable yields. An even lower yield was reported in the final step of the published synthesis of compound 3. (Brown et al., supra). Individual synthetic steps are described in greater detail below.

### 1(c) Bis(4-methoxyphenyl)amine (compound 5)

Compound 5 was prepared in 75% yield according to a standard literature procedure (Zhang et al., Org. Chem. 2005, 70:5164-73). A mixture of 4-anisidine (2.46 g, 20 mmol), 4-iodoanisole (6.12 g, 30 mmol), K₂CO₃ (5.53 g, 40 mmol), CuI (0.38 g, 2 mmol), and L-proline (0.46 g, 4 mmol) in DMSO (30 mL) was heated at 90° C. for 48 h under dry conditions. The cooled mixture was partitioned between water and ethyl acetate. The organic layer was separated, and the aqueous layer was extracted with ethyl acetate. The combined organic layers were washed with brine, dried (MgSO₄), and concentrated under reduced pressure. The residue obtained was purified by column chromatography (silica gel; Et₂O-hexane, 1:4) to give 5 (3.43 g, 15.0 mmol; 75% yield) as a crystalline material: mp 102-103° C. (lit. 102-103° C. (McNulty at al., Eur. J. Org. Chem. 2007, 9:1423-28); 99.5-101.5° C. (Wolfe et al., J. Org. Chem. 2000, 65:1158-74)); ¹H NMR (CDCl₃) δ6.98 (d, J=8.8 Hz, 4H), 6.86 (d, J=8.8 Hz, 4H), 5.33 (s, exch, 1H), 3.81 (s, 6H); ¹³C NMR (CDCl₃) δ 154.3, 138.0, 119.6, 114.8, 55.7; HRMS (EI) calcd for C₁₄H₁₅NO₂ (M⁺) 229.1103. found 229.1106.

### 1(d) N-(4-Methoxyphenyl)-5-methoxyisatin (compound 6)

A solution of 5 (3.21 g, 14.0 mmol) in dichloromethane (CH₂Cl₂, 40 mL) was added drop-wise to a stirred, refluxing solution of oxalyl chloride (3.53 g, 28.0 mmol) in CH₂Cl₂ (70 mL). The mixture was heated under reflux for 1 h, and the excess oxalyl chloride and CH₂Cl₂ were then removed under reduced pressure. To the residue, CH₂Cl₂ (100 mL) was added, followed by anhydrous AlCl₃ (4.32 g, 32.4 mmol) portion-wise over 10 min. The mixture was heated under reflux for 1 h. The solvent was removed under reduced pressure, and to the residue was added dilute HCl (40 mL of about 1 M). The mixture was stirred for 30 min and was then extracted with CHCl₃ (3-30 mL). The extracts were combined and dried (MgSO₄), and the solvent was removed under reduced pressure. The solid obtained was washed with CH₂Cl₂ to give pure 6 (2.78 g, 9.81 mmol; 70%): mp 203-205° C.; ¹HNMR (CDCl₃) δ 7.34 (d, J=9.0 Hz, 2H), 7.25 (d, J=2.8 Hz, 1H), 7.13 (dd, J=2.8 and 8.6 Hz, 1H), 7.07 (d, J=9.0 Hz, 2H), 6.79 (d, J=8.6 Hz, 1H), 3.88 (s, 3H), 3.84 (s, 3H); ¹³C NMR (CDCl₃) δ 183.5, 159.5, 157.7, 156.7, 146.1, 127.3, 125.5, 125.0, 117.8, 115.2, 112.3, 109.1, 56.0, 55.6; HRMS (APCI) calcd for C₁₆H₁₄NO₄ (MH+) 284.0923. found 284.0935.

### 1(e) 2,7-Dimethoxyacridine-9-carboxylic Acid (compound 7)

A mixture of 6 (1.42 g, 5.00 mmol) and KOH (7.00 g, 125 mmol) in water (70 mL) was refluxed for 72 h. The resulting mixture, after cooling, was poured into a mixture of concentrated HCl (15 mL) and ice (30 g). The yellow solid obtained was collected by filtration, washed with water and CH₂Cl₂, and then dried in a vacuum oven at 60° C. overnight to give 7 (1.35 g, 4.76 mmol; 95%): mp 192-193° C.; ¹H NMR (DMSO-d₆) δ 8.32 (d, J=9.4 Hz, 2H), 7.75 (dd, J=2.6 and 9.4 Hz, 2H), 7.29 (d, J=2.6 Hz, 2H), 3.97 (s, 6H). The material was highly insoluble in solvents, and the ¹³C NMR spectrum was not recorded. HRMS (EI) calcd for C₁₆H₁₃NO₄ (M+) 283.0845. found 283.0845.

### 1(f) 2,6-Dibromophenyl 2,7-Dimethoxyacridine-9-carboxylate (compound 8)

A mixture of 7 (1.21 g, 4.27 mmol) and freshly distilled thionyl chloride (20 mL, 69 mmol) was refluxed under anhydrous conditions for 3 h. The excess thionyl chloride was removed under reduced pressure to leave an orange solid. 2,6-Dibromophenol (1.33 g, 5.30 mmol) was dissolved in pyridine (20 mL) by heating at 70° C., cooled, and transferred into the flask containing the acid chloride. The mixture was heated at 70° C. overnight. The solvent was removed under reduced pressure, and the residue obtained was extracted with CH₂Cl₂. The extract was concentrated, and the residue obtained was purified by column chromatography (silica gel; Et₂O-hexane, 1:3) to give 8 (1.48 g, 2.86 mmol; 67% yield): mp 245-247° C.; ¹H NMR (CDCl₃) δ 8.17 (d, J=9.4 Hz, 2H), 7.89 (d, J=2.6 Hz, 2H), 7.74 (d, J=8.1 Hz, 2H), 7.48 (dd, J=2.6 and 9.4 Hz, 2H), 7.18 (t, J=8.1 Hz, 1H), 4.02 (s, 6H); ¹³C NMR (CDCl₃) δ 164.0, 158.9, 146.7, 144.25, 132.9, 131.8, 128.9, 128.2, 125.0, 124.3, 117.9, 101.3, 56.1; HRMS (EI) calcd for C₂₂H₁₅NO₄⁷⁹Br₂ (M+) 514.9368. found 514.9367.

### 1(g) Succinimidyl 4-Iodobutanoate

Succinimidyl 4-iodobutanoate was prepared in 84% yield according to a standard literature procedure. (Brown et al., Org. Biomol. Chem. 2009, 7:386-94). 4-Iodobutanoic acid (0.48 g, 2.24 mmol) in dry THF (10 mL) was cooled to 0° C., and N-hydroxysuccinimide (0.25 g, 2.17 mmol) in dry THF (2 mL) and dicyclohexylcarbodiimide (DCC; 0.52 g, 2.50 mmol) in THF (4 mL) were added, successively. The mixture was stirred at 0° C. for 3 h, and then at room temperature overnight. The mixture was filtered, and the filtrate was concentrated under reduced pressure. The residue obtained was purified by column chromatography (silica gel; Et₂O-hexane, 1:2) to give succinimidyl 4-iodobutanoate (0.59 g, 1.89 mmol; 84% yield) as a colorless solid: mp 86-87° C. (lit. 86-87° C. (Brown et al., supra)); ¹H NMR (CDCl₃) δ3.03 (t, J=7.0 Hz, 2H), 2.62 (s, 4H), 2.53 (t, J=7.0 Hz, 2H), 2.05 (apparent quintet, J=7.0 Hz, 2H); HRMS (APCI) calcd for C₈H₁₁NO₄I (MH⁺) 311.9733. found 311.9736.

### 1(h) 9-(2,6-Dibromophenoxycarbonyl)-2,7-dimethoxy-10-(3-succinimidyloxycarbonylpropyl)acridinium Iodide (compound 4)

A well-mixed mixture of 8 (50 mg, 0.091 mmol) and succinimidyl 4-iodobutanoate (50 mg, 0.16 mmol) was heated in an oil bath (150° C.) for 3 h under nitrogen. The mixture turned to dark brown, and acetonitrile (3×1 mL) was used to extract the product. The crude product was precipitated with diethyl ether (10 mL), then subjected to extensive washing with diethyl ether and diethyl ether/CH₂Cl₂ mixture (5/1 by volume) until TLC showed that all the starting materials were removed, leaving 4 as a brownish solid (3 mg, 0.0036 mmol; 4%). The washings were evaporated under reduced pressure. The residue was subjected to the same reaction procedure again to give further 4 (1 mg), and the cycle was repeated yet again to give further 4 (1 mg). The total yield of 4 was 6.6%: mp 159-162° C.; ¹H NMR (CD₃CN) δ 8.64 (d, J=9.5 Hz, 2H), 8.07 (dd, J=2.7 and 9.5 Hz, 2H), 7.98 (d, J=2.7 Hz, 2H), 7.90 (d, J=8.2 Hz, 2H), 7.36 (t, J=8.2 Hz, 1H), 5.44 (t, J=7.0 Hz, 2H), 4.08 (s, 6H), 3.16 (t, J=7.0 Hz, 2H), 2.85 (s, 4H), 2.57 (m, 2H); HRMS (ES+) calcd for C₃₀H₂₅N₂O₈⁷⁹Br₂ ([M-I]+) 698.9978. found 698.9982.

### Example 2

### Selection of Target and Probe Sequences

Four target sequences were selected (Table 1: SEQ ID NOs:1-4, 16, 17; Table 2) as representatives of two different, broad microbial groups found in environmental samples such as seawater (Escherichia coli and Candida albicans) (Hartz et al., J. Environ. Qual. 2008, 37:898-905; Papadakis et al., Water Res. 1997, 31:799-804) or of two pathogens that may occur individually or simultaneously in human urogenital swab or urine specimens (Chlamydia trachomatis and Neisseria gonorrhoeae). (Johnson et al., Clin. Chem. 2001, 47:760-63). In order to mimic more closely the natural nucleic acid materials, the synthetic target oligonucleotide sequences exceeded the length of the part for which complementary sequences in probes would be constructed (Table 1: SEQ ID NOs:5-8; Table 2: central part in each target sequence). These targets were synthesized on automated oligonucleotide synthesizers by established methods using phosphoramidite monomers, then purified and quantified prior to use, as described in greater detail in Example 3. Target sequences denoted as CtrB1447-1470(−), NgoA128-150(−) in Table 2 contain a 3′-terminal deoxycytidine residue with a reversed 3′→5′ polarity and directly correspond to SEQ ID NOs:16 and 17 in Table 1. SEQ ID NOs: 3 and 4 do not contain this residue because it is not believed to play a role in target-probe hybridization.

Selected probe sequences (Table 1: SEQ ID NOs: 9-15; Table 2) were based on earlier fluorescent probes (Browne, K. A., J. Am. Chem. Soc. 2005, 127:1989-94) used in detection of pan-bacterial and pan-fungal species or were derived from earlier probe sequences (U.S. Pat. No. 5,693,468) used to detect C. trachomatis and N. gonorrhoeae specifically but with self-complementary arm sequences added to each pair of termini. The probes were also synthesized on automated oligonucleotide synthesizers by established methods using phosphoramidite monomers. Several different pairs of stem arms were incorporated into the oligonucleotide sequences, all of which included GC clamps at their termini to maximize the affinity of the stem arms for each other. The probe sequences were furnished with one of two quenching moieties: (i) a 3′-BHQ2 unit incorporated as part of the oligonucleotide synthesis; (ii) an iodoacetyl-methyl red labeling reagent (U.S. Pat. No. 7,169,554; U.S. Pub. No. 2007/0166759 A1; Brown et al., Org. Biomol. Chem. 2009, 7:386-94) allowed to react with (after reduction) a 3′-disulfide group incorporated as part of the oligonucleotide synthesis. The probe oligonucleotides were synthesized with a terminal protected 5′-hexylamino phosphoramidite at the end of the oligonucleotide synthesis sequence. After deprotection, the chemiluminophore reagents were used to label the freed 5′—NH₂ group as described in detail in Example 4. The seven probes produced were purified and quantified prior to use.

TABLE 2
Nucleic acid sequences for chosen targets and the probes developed for them.

| | Nucleic acid | |
|---|---|---|
| Name | backbone | Sequence and substituents |
| EcoB1932-1947(−)HICS16 EcoB1932-1947(−)HICS17 EcoB1932-1947(−)HICS18 EcoB1921-1958(+) | DNA/OMe DNA/OMe DNA/OMe RNA/DNA | 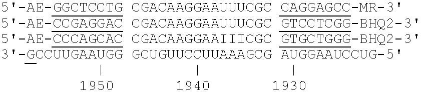 |
| CalA1185-1206(−)HICS86 CalA1185-1206(−)HICS87 CalA1174-1217(+) | DNA DNA RNA/DNA | 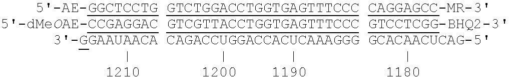 |
| CtrB1452-1645(+)HICS51 CtrB1447-1470(−) | OMe OMe/DNA | 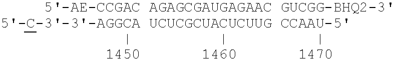 |
| NgoA133-145(+)HICS62 NgoA128-150(−) | OMe OMe/DNA | 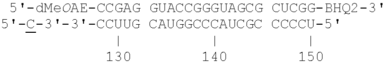 |

In Table 2, names indicating probe sequence ranges corresponding to target sequence ranges are denoted by three letter abbreviations (Genus, species) of the reference sequences, a letter indicating whether the small (A) or large (B) rRNA subunit, and the target sequence range (probes have an additional unique designation, e.g., HICS16, from a longer list of previous probes). Eco-series sequences (U.S. Pat. No. 6,821,770) are pan-bacterial probes and targets to/from the Escherichia coli O157:H7 23S rRNA reference sequence (NCBI Accession No. E16366). Cal-series sequences (U.S. Pat. No. 6,821,770) are pan-fungal probes and targets to/from the Candida albicans 18S rRNA reference sequence (NCBI Accession No. E15168). Ctr-series sequences are probes and targets to/from TMA amplicon sequences of Chlamydia trachomatis 23S rRNA (NCBI Accession No. AM884176). Ngo-series sequences are probes and targets to/from TMA amplicon sequences of Neisseria gonorrhoeae 16S rRNA (NCBI Accession No. NC_{—}002946). Names of target sequences are provided in bold characters. Nucleic acid backbone sugar structures used for corresponding sequences: DNA, deoxyribose; RNA, ribose; OMe, 2′-O-methylribose (OMe oligonucleotides have similar affinities for complementary RNA sequences (Lesnik et al., Biochemistry 1998, 37:6991-97) while being more resistant to nucleases than RNA oligonucleotides (Sproat, et al., Nucleic Acids Res. 1989, 17:3373-86)). Underlined probe sequences were composed of DNA, remaining probe sequences are composed of OMe. AE and dMeOAE are N-linked chemiluminophores introduced using labeling reagent 3 or 4, respectively. BHQ2 and MR are Black Hole Quencher-2 and methyl red quenching moieties, respectively.

### Example 3

### Synthesis of Oligonucleotides

Oligonucleotides were synthesized in-house using an Expedite model 8909 nucleic acid synthesis system (PerSeptive Biosystems, now part of Life Technologies Corporation, Carlsbad, Calif.). The syntheses of the probe oligonucleotides utilized substituted 500 Å controlled pore glass (CPG) substrates packed in automated synthesizer columns. Deoxy CPG were used for RNA target syntheses, resulting in 3′-deoxyribonucleotides on these oligonucleotides. Probe sequences incorporating a 3′-terminal BHQ2 unit started with Black Hole Quencher-2 attached to CPG via a glycolate linker and with a dimethoxytrityl (DMT) protecting the terminal hydroxyl (part no. CG5-5042G, Biosearch Technologies, Inc., Novato, Calif.), and sequences destined for eventual conjugation to 2-(4-dimethylaminophenylazo)-N-[2-(2-iodoacetylamino)ethyl]-benzamide (iodoacetyl-methyl red) to incorporate the quenching agent started with 1-O-dimethoxytrityl-propyl-disulfide, 1′-succinyl-[long chain alkylamino]-CPG (part no. 20-2933, Glen Research, Sterling, Va.). 6-(4-Monomethoxytritylamino)hexyl (2-cyanoethyl)(N,N-diisopropyl) phosphoramidite (part no. 10-1906, Glen Research, Sterling, Va.) was used to functionalize probe oligonucleotides with 5′-terminal amine linker arms. The dimethoxytrityl group was removed (as trityl groups were for subsequently incorporated phosphoramidites) by machine-automated detritylation with trichloroacetic acid in dichloromethane according to vendor recommendations. After completion of their syntheses, oligonucleotides were cleaved from the CPG column under standard conditions with ammonium hydroxide, followed by purification by standard polyacrylamide gel electrophoretic separation as previously described. (Browne, K. A., J. Am. Chem. Soc. 2005, 127:1989-94).

### Example 4

### Labeling of Oligonucleotides

The oligonucleotides were deprotected to liberate the terminal amino group according to vendor recommendations. The amino groups were then labeled through the succinimidyl groups of 9-(2,6-dibromophenoxycarbonyl)-10-(3-succinimidyloxycarbonylpropyl)acridinium iodide (3) or 9-(2,6-dibromophenoxycarbonyl)-2,7-dimethoxy-10-(3-succinimidyloxycarbonylpropyl)acridinium iodide (4). (U.S. Pat. No. 7,169,554; U.S. Pub. No. 2007/0166759 A1; Brown et al., Org. Biomol. Chem. 2009, 7:386-94). The 3′-disulfides were reduced according to vendor recommendation with dithiothreitol and coupled with iodoacetyl-methyl red according to the literature procedure. (Id.) The fully labeled oligonucleotides were purified by reversed phase HPLC as previously described. (Browne, K. A., supra). The structures of the oligonucleotides (labeled or not) were confirmed by MALDI-TOF mass spectroscopy, and their concentrations were determined by conventional absorption spectroscopy using a DU 640B spectrophotometer (Beckman Coulter, Inc., Brea, Calif.).

### Example 5

### Testing of Chemiluminescent Probes

Substituents on the phenoxide leaving group of an AE have major implications for the rate of the chemiluminescent reaction, but all of our probes had the same leaving group, chosen to provide a balance for initiation of the reaction at moderate pH. However, substituents on the acridinium ring can influence the rate of reaction (Nelson et al., Biochemistry 1996, 35:8429-38), so it was important also to investigate the time courses of emissions and advantageously use differences found to enhance discrimination of emissions. Therefore, several additional characterizations were undertaken, namely, time-resolved spectrography and time-resolved chemiluminescent intensity measurements for individual probes in unhybridized and fully hybridized forms and dose-response measurements for both individual probes and mixtures of probes in the presence of a single or two different target oligonucleotides.

5(a) Time-Resolved Spectrography

In order to understand how 2,7-dimethoxy substitution of the AE changes photonic output compared to the unsubstituted AE, time-resolved, spectrographic images of HICS probe chemiluminescent emissions were acquired on a low light, echelle-type SE200 spectrograph (Optomechanics Research Inc., Vail, Ariz.) using KestrelSpec software (Catalina Scientific Corp., Tucson, Ariz.). The images were converted to spectrograms at 1 nm resolution and then smoothed (4%) with a locally weighted scatter plot smoothing algorithm (Excel Add-In, Peltier Technical Services). (Cleveland, W. S., J. Am. Stat. Assoc. 1979, 74:829-36; Cleveland et al., J. Am. Stat. Assoc. 1988, 83:596-610). An amount of 50 pmol/100 μL of AE-bearing probes or 300 pmol/100 μL of 2,7-dimethoxyAE-linked probes, either alone or together with 50 or 300 pmol/100 μL of the corresponding specific synthetic target (Table 2), was allowed to incubate for 15 min at 60° C. in a low pH, surfactant-containing 1× hybridization reagent (indicating hybridization or pre-detection concentrations; 95 mM succinic acid, 1.5 mM ethylenediamine-N,N,N′,N′-tetraacetic acid, 1.5 mM ethylene glycol-bis(2-aminoethylether)-N,N,N′,N′-tetraacetic acid, 312 mM lithium dodecyl sulfate, 125 mM LiOH, pH 5.2) (U.S. Pat. No. 7,169,554; U.S. Pub. No. 2007/0166759 A1) and cooled for at least 15 min at room temperature. These conditions allowed probe loop hybridization to target sequences to occur in the solutions containing target and probe stem hybridization to occur in solutions without target. Chemiluminescence from 100 μL of each of the solutions (probes incubated with or without complementary target) was initiated by a single 100 μL injection of 240 mM H₂O₂/2 M Tris-HCl, pH 9.0, solution, and data collection was started immediately (for 25×0.4 s or 5 s intervals separated by 0.37 s pauses for the AE-linked or 2,7-dimethoxyAE-linked probes, respectively).

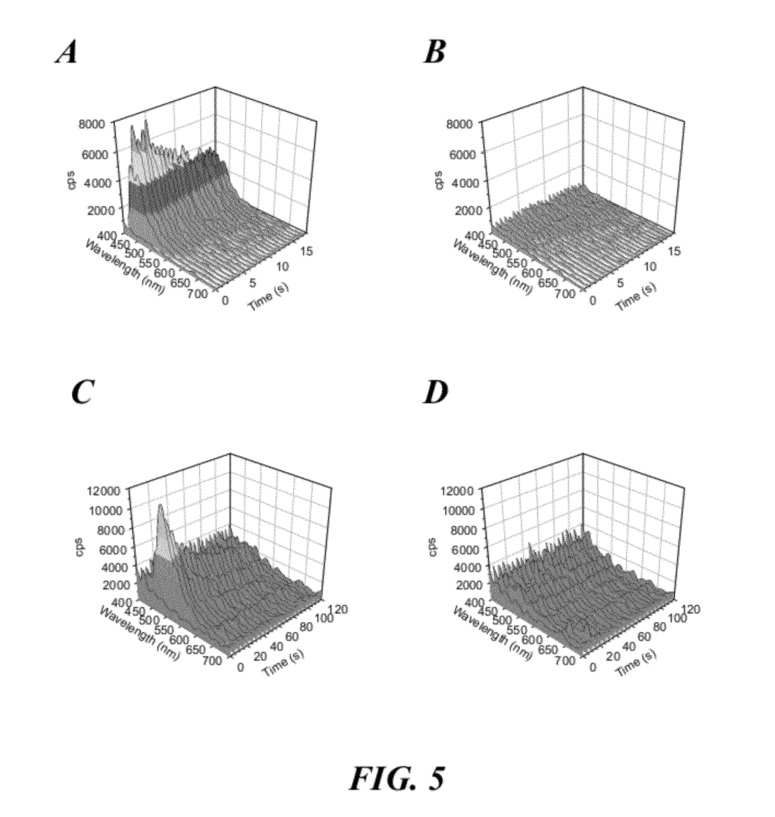

As evidenced from the time-resolved spectrograms of the N-alkylacridone from probe EcoB1932-1947(−)HICS18 hybridized to an equimolar amount of its complementary target (EcoB1921-1958(+), FIG. 5A), chemiluminescence increased rapidly in the 400-500 nm range (with smaller contributions from 500-540 nm) during the first 1-1.5 s (compare the initial baseline-level spectrogram at t=0 with the two immediately subsequent spectrograms), followed by a slower decay for at least 15 s (spectrograms 3-23). In the absence of target, chemiluminescence from this probe was low over the entire time course (FIG. 5B), similar to the chemiluminescence of the buffer in the absence of probe (data not shown). Double emission maxima at about 425 and 448 nm were readily evident in the EcoB1932-1947(−)HICS18 plus target chemiluminescence spectrograms, consistent with N-alkylacridone fluorescence peaks at about 430 and 450 nm that we have observed in water (Li, Z., Ph.D. Thesis, supra) and peaks at 430 and 443 nm previously reported in aqueous ethanol. (McCapra et al., Photochem. Photobiol. 1965, 4:1111-21). The leading edges of the AE spectrograms were steep, while the trailing edges (higher wavelengths) were somewhat broad beyond the second peak. The magnitudes of chemiluminescent spectrograms from the N-alkyldimethoxyacridone of probe CalA1185-1206(−)HICS87 hybridized to an equimolar amount of its complementary target (CalA1174-1217(+), FIG. 5C) increased rapidly in the 440-550 nm range [compare the initial spectrogram at t=0 with the immediately subsequent spectrogram; chemiluminescence from this probe in the absence of target was low over the entire time course (FIG. 5D), again comparable to buffer alone (data not shown)].

Notably, chemiluminescence from the N-alkyldimethoxyacridone decayed much more slowly than the N-alkylacridone (spectrograms 3-23), nearing background levels after 120 s. Single peak emissions were at about 483 nm, very nearly the same as the 485 and 484 nm peaks found for related compounds in water (Batmanghelich et al., supra) and dimethylformamide (Natrajan et al., Anal. Biochem. 2010, 406:204-13), respectively. However, the present results also showed a pronounced shoulder at about 525 nm. This shoulder from the N-alkyldimethoxy-acridone was separated substantially more than the double peaks from the unsubstituted N-alkylacridone (about 40 nm versus 23 nm, respectively). The leading and trailing edges of the N-alkyldimethoxyacridone were more symmetric than those of N-alkylacridone. The emission rise and fall was uniform across spectrograms of both HICS probes, without any isosbestic points, supporting emitted chemiluminescence arising from a single species. Although partially overlapping, the emissions from these two acridinium esters were substantially separated below about 450 nm and above about 550 nm. In addition to defining the wavelength ranges expected from probes with these two labels, the spectrography results suggested initial ranges for quantifying time-based constants for the different probes.

5(b) Time-Resolved Chemiluminescence

Constants for total chemiluminescent emissions from HICS probes were determined after hybridization to excess complementary target oligonucleotides. Preliminary hybridization time courses indicated that incubation for 40 min at 60° C. and 10 min at room temperature was sufficient to maximize signal output from each probe under the conditions employed (data not shown). Chemiluminescent emission time courses were acquired on a Gen-Probe Incorporated LEADER HC+ luminometer. Solutions containing 0.5 pmol/100 μL probe with or without 1 pmol/100 μL specific synthetic target in 1× hybridization reagent plus 100 μL of silicone oil were mixed by vortexing in 12 mm×75 mm polypropylene tubes and allowed to incubate in a 60° C. water bath for 40 min to facilitate hybridization of probes and targets (when present). After the mixtures had cooled to room temperature for at least 10 min, chemiluminescent emissions were initiated by a 200 μL injection of Detect 1 solution (240 mM H₂O₂/1 mM HNO₃) followed by a 2 s pause and then a 200 μL injection of Detect 2 solution (2 M Tris-HCl, pH 9.0), and data collection was started after the first 40 ms mixing time and continued for at least 5τ_{1/2} of the apparent first order rate constant k_{b} (10-500 s total) without inter-interval delays. Final pH after detection was always about 9.0.

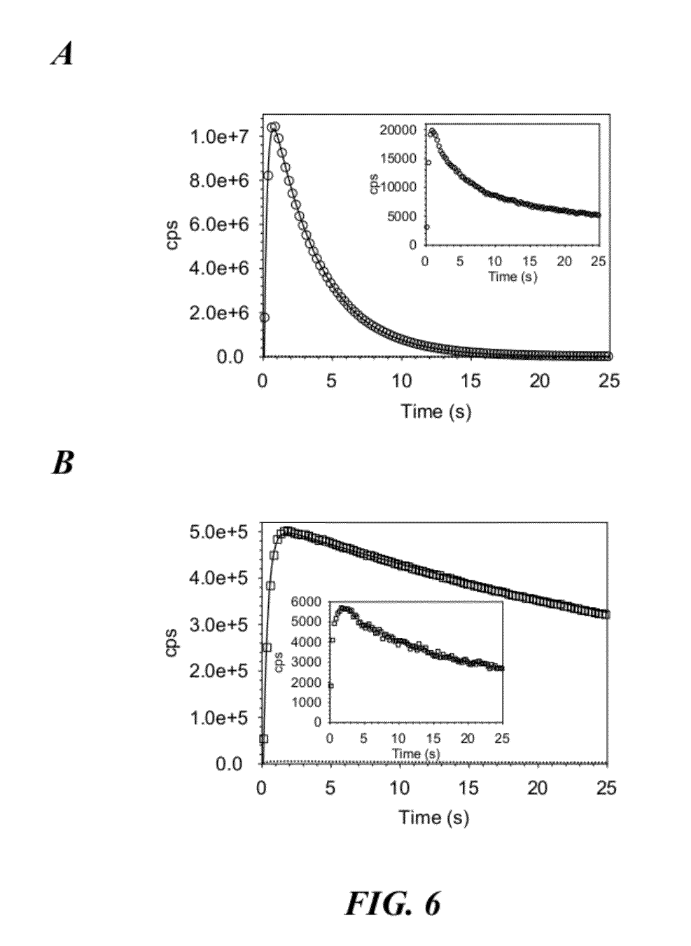

Plots of counts per second (cps) versus time indicated a rapid increase in emissions to a maximum followed by a slow decrease trending toward the baseline (FIG. 6). Background (probe but no target) emissions followed similar time course patterns but with substantially attenuated magnitudes (FIG. 6, insets; note the different scales compared to those in the presence of targets). Chemiluminescent constants were calculated as described below. Interpretation of the kinetic constant kₐ may be somewhat limited by the hardware and firmware configurations of the luminometer.

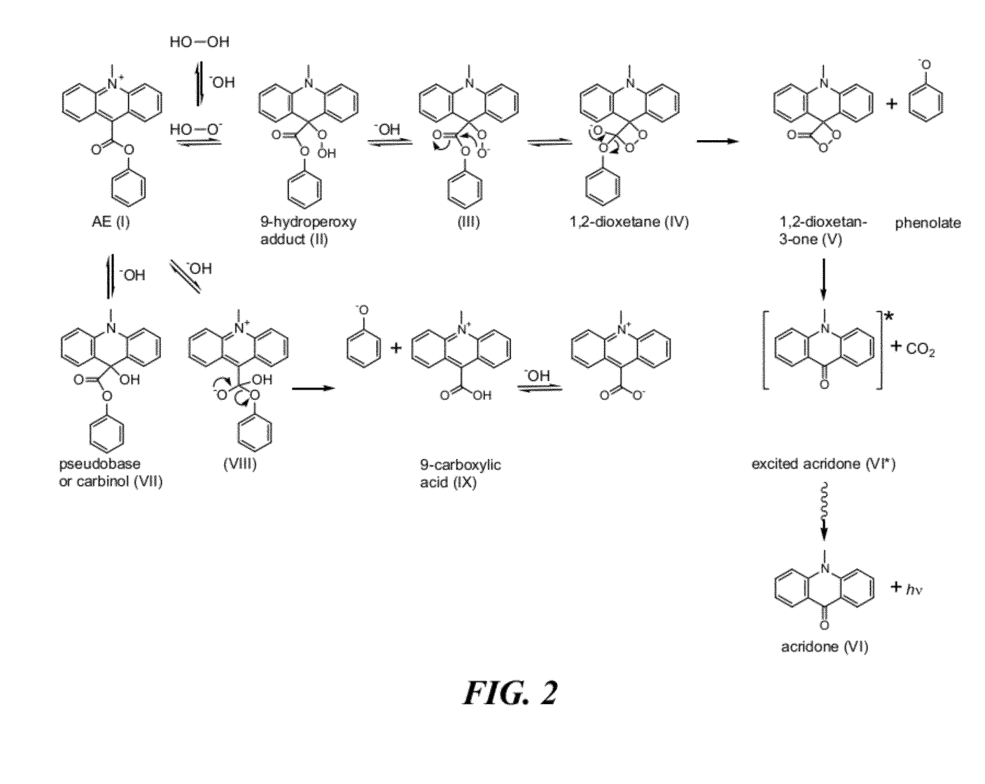

Multiple series of chemical events occur upon addition of alkaline peroxide to AEs (FIG. 2). In order to be able to compare different probes quantitatively, it was necessary to simplify the kinetic scheme for the reactions leading to the increase and decrease in chemiluminescence and then to test this scheme against the observed data. AEs are chemically similar to lucigenin in that both classes of compounds emit light through the same type of chemiluminescent intermediate, an excited N-alkylacridone. In the reaction of lucigenin with basic oxygen, it has been demonstrated that despite photon emission from the excited N-methylacridone being the result of a minor reaction path, a pair of apparent first-order reactions for the formation and decay of chemiluminescent emissions accounted for their data based on a pair of competing, parallel reactions. (Maskiewicz et al., J. Am. Chem. Soc. 1979, 101:5355-64). We adapted the kinetic scheme of Maskiewicz et al. for our needs (Eq. 1):

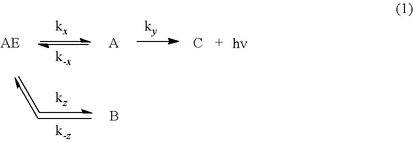

This scheme only considers rate limiting formation of a pre-luminescent intermediate A from the AE label prior to initiation of chemiluminescence, rapid formation of a post-luminescent product C with emission of light, and a competing reaction to give a non-chemiluminescent product B. If the dark process competes effectively with the process leading to intermediate A, the concentration of A decreases rapidly from its peak and the overall emission process is of short duration. If the dark process is much slower than the process leading to intermediate A, then the emission profile reflects more closely the rate of formation of A and the overall emission process is of longer duration. Data in plots of light emission versus time (e.g., FIG. 6) were fitted to a linear combination of exponential functions based on this scheme (Eq. 2, where a and b are pre-exponential factors for kₐ and k_{b}, respectively; kₐ=kₓ−k₋ₓ is the apparent first-order rate constant for the rate determining step for increase in light intensity; k_{b}=k_{z}−k_{-z} is the apparent first-order rate constant for the decrease in light intensity; k_{y} is the constant for the rapid emission of light as the excited intermediate decays to its ground state; k_{y}>k_{b}; and t is time in seconds). The constants kₐ and k_{b}, specific activities, and signal-to-background ratios are compared along with other parameters in Table 3.

cps=+a(−exp^{-kat})+b(exp^{-kbt}) (2)

TABLE 3
Chemiluminescent emission constants for HICS probes.^{a}

| | Time- | | | | | | | | | |
|---|---|---|---|---|---|---|---|---|---|---|
| | to-peak | | | | | | | | | |
| Name | (s) | | kₐ (s⁻¹) | | k_{b} (s⁻¹) | | S.A.^{b} (Σcps/pmol) | | S/B^{b} | |
| EcoB1932-1947(−)HICS16 | 1.3 | [16] | 2.57 ± 0.14 | [18] | 0.157 ± 0.004 | [16] | (3.93 ± 0.22) × 10⁷ | [16] | 4.60 ± 0.33 | [16] |
| EcoB1932-1947(−)HICS17 | 0.63 | [16] | 4.53 ± 0.15 | [18] | 0.339 ± 0.019 | [16] | (2.08 ± 0.06) × 10⁸ | [16] | 150 ± 6 | [16] |
| EcoB1932-1947(−)HICS18 | 0.70 | [6] | 3.99 ± 0.07 | [6] | 0.291 ± 0.008 | [14] | (3.34 ± 0.09) × 10⁸ | [14] | 285 ± 10 | [14] |
| CalA1185-1206(−)HICS86 | 1.0 | [18] | 3.49 ± 0.17 | [20] | 0.141 ± 0.003 | [18] | (3.74 ± 0.08) × 10⁸ | [18] | 73.3 ± 2.8 | [18] |
| CalA1185-1206(−)HICS87 | 1.8 | [16] | 2.92 ± 0.27 | [18] | 0.0188 ± 0.0004 | [16] | (2.27 ± 0.04) × 10⁸ | [2] | 93.6 ± 2.5 | [2] |
| CtrB1452-1465(+)HICS51 | 0.75 | [14] | 2.11 ± 0.09 | [14] | 0.924 ± 0.019 | [14] | (1.72 ± 0.04) × 10⁸ | [14] | 276 ± 11 | [14] |
| NgoA133-145(+)HICS62 | 2.1 | [14] | 2.86 ± 0.21 | [17] | 0.00735 ± 0.00032 | [14] | (1.67 ± 0.07) × 10⁸ | [3] | 40.4 ± 1.8 | [3] |

^{a}Values are means ± standard deviations. Numbers in brackets are numbers of replicates. kₐ and k_{b} are from non-linear best fits to Eq. 2 from at least 5 × first order half lives (τ_{1/2}) of each constant.
^{b}Specific activities (S.A.) are from the summed cps from 5 × τ_{1/2} of the decay process divided by the picomoles of probe, and signal-to-background ratios (S/B) are from the summed cps of 5 × τ_{1/2} of the decay process for probe-target hybrids divided by the same sum for probes alone.

The emission increase constant (kₐ in Eq. 2) varied little more than about 2-fold despite the different probes varying in terms of target-specific sequence, ribose backbone, labels and quenching moieties (Table 2). This is consistent with all of the probes containing the same 2,6-dibromophenoxy leaving group. The constant for decreasing emission (k_{b}), on the other hand, varied about 125-fold among probes, leading to τ_{1/2} for decay in light emission ranging from 0.75 to 94 s. However, for a given AE label-quencher combination, k_{b} varied only up to about 3-fold (e.g., 1.1-fold for probes containing AE and methyl red; 3.2-fold for probes containing AE and BHQ2; and 2.5-fold for probes containing 2,7-dimethoxyAE and BHQ2). The average value of k_{b} for the two probes bearing a 2,7-dimethoxyAE unit was almost 30 times lower than the average value for all probes bearing an AE unit without modifications at acridinium positions 1-8, indicating that the dark reaction was much less significant for 2,7-dimethoxyAEs than for unmodified AEs. The time to peak, a balance of formation and decay of light emissions, varied about 3.3-fold among the probes.

Not surprisingly, the probes with the longest times to peak and smallest k_{b} values were those comprising 2,7-dimethoxyAE units, which clearly exhibited emission over longer periods of time (FIG. 6). Probes bearing unsubstituted AE units and quenchers with a wavelength absorption profile that was well matched with the emitter wavelength profile (e.g., EcoB1932-1947(−)HICS16, CalA1185-1206(−)HICS86) had longer times-to-peak, smaller values of k_{b}, and lower S/B than the probes for which the quencher absorption profile was less well matched to the emitter profile (e.g., EcoB1932-1947(−)HICS17, EcoB1932-1947(−)HICS18, CtrB1452-1465(+)HICS51). With the exception of the pan-bacterial probe with a methyl red quencher, which showed an SA of only about 4×10⁷ Σcps/pmol and a S/B of less than 5, SA and S/B of all probes were high, exceeding 10⁸ Σcps/pmol and 40, respectively.

Chemiluminescent emission constants were used to select probe constructs with preferred attributes. For the current studies, the Ctr/Ngo pair of distinguishable probes and their target was not chosen as a model system because of the very long data collection that would be required for NgoA133-145(+)HICS62 (5τ_{1/2}≈7.8 min). The only pan-fungal probe that could be clearly distinguished from the pan-bacterial AE-labeled probes was 2,7-dimethoxyAE-labeled CalA1185-1206(−)HICS87. From the pan-bacterial probes, the SA and S/B from EcoB1932-1947(−)HICS16 were substantially lower than from EcoB1932-1947(−)HICS17 or EcoB1932-1947(−)HICS18. Preliminary experiments with EcoB1932-1947(−)HICS17 and CalA1185-1206(−)HICS87 resulted in increased quenching of each other as a function of increased specific probe-target hybrid, suggesting interactions between the arms of these two different probes in the open state (data not shown). Therefore, EcoB 1932-1947(−)HICS18 and CalA1185-1206(−)HICS87 were selected for demonstrating utility for simultaneous detection, discrimination, and quantification of two targets in individual samples.

5(c) Wavelength-Resolved Chemiluminescence

Since the probes were designed and shown to be distinguishable within a mixture by differences in wavelengths of emission, it was necessary to adapt a luminometer to distinguish multiple wavelengths simultaneously. This was achieved for two probe systems by incorporation of a second PMT into a luminometer and separately attenuating the pair of PMTs with two different filters, one of which allowed light of relatively short wavelength (≦450 nm) to pass while the other allowed only light of longer wavelength (≧550 nm) to pass. In an attempt to emulate assay conditions for simultaneous, direct detection of bacteria and/or fungi in an environmental sample, constant amounts of both EcoB1932-1947 (−) HICS18 and CalA1185-1206(−)HICS87 were allowed to hybridize to increasing amounts of one, the other, or both target nucleic acids in a mixture of seawater and hybridization reagent.

Chemiluminescent emission time courses from 0.2 pmol/100 μL of both EcoB1932-1947(−)HICS18 and CalA1185-1206(−)HICS87 probes in hybridization reagent were acquired simultaneously on a luminometer modified for dual wavelength-range detection. This luminometer was equipped with two high-count PMT modules (28 mm diameter, headon, bi-alkali cathode, peak cathode radiant sensitivity at 420 nm; Hamamatsu Photonics, Hamamatsu City, Japan) on opposite sides of and directed toward a light-tight detection chamber fitted with injector tubing from two reagent pumps. Filters (25.4 mm diameter; 417/60 BrightLine bandpass filter [transmits about 95% of light from 385 to 450 nm] from Semrock, Inc., Rochester, N.Y. for AE, and OG550 cut-on filter [transmits about 90% of light from 550 to 700 nm] from Newport Corporation, Irvine, Calif. for 2,7-dimethoxyAE) were fitted between PMT 1 and PMT 2, respectively, and the detection chamber. Custom Visual Basic software controlled reagent injections and chemiluminescent data acquisition from the two PMT channels. Hybridizations were performed substantially as for Time-Resolved Chemiluminescence (above) except an amount of 50 μL of 0 or 200 amol to 200 fmol/50 μL targets in seawater (0.2 μm filtered; Scripps Institution of Oceanography Pier, La Jolla, Calif.) was mixed with 50 μL of 2× hybridization reagent containing 0.5 pmol/50 μL of both probes. Chemiluminescence was initiated by a 200 μL injection of Detect 1 solution followed by a 2 s pause, then a 200 μL injection of Detect 2 solution, and data collection was started immediately from the dual wavelength luminometer for 230×0.8 s intervals (184 s total) without inter-interval delays. Final pH after detection was always about 9.0.

As is evident from FIG. 5, some longer wavelength emissions from EcoB1932-1947(−)HICS18 overlapped with some shorter wavelength emissions from CalA1185-1206(−) HICS87. Such overlaps could be problematic for distinguishing emissions at higher concentrations of each target. However, emission decays from EcoB1932-1947(−)HICS18 were rapid after a high peak emission, while those from CalA1185-1206(−)HICS87 were considerably slower and had a substantially lower maximum peak intensity (FIG. 6 and Table 3). Taking advantage of wavelength separations as well as these timing differences facilitated considerable reduction in overlapping emissions from these probes at high concentrations without detrimental reduction in specific target signal intensities.

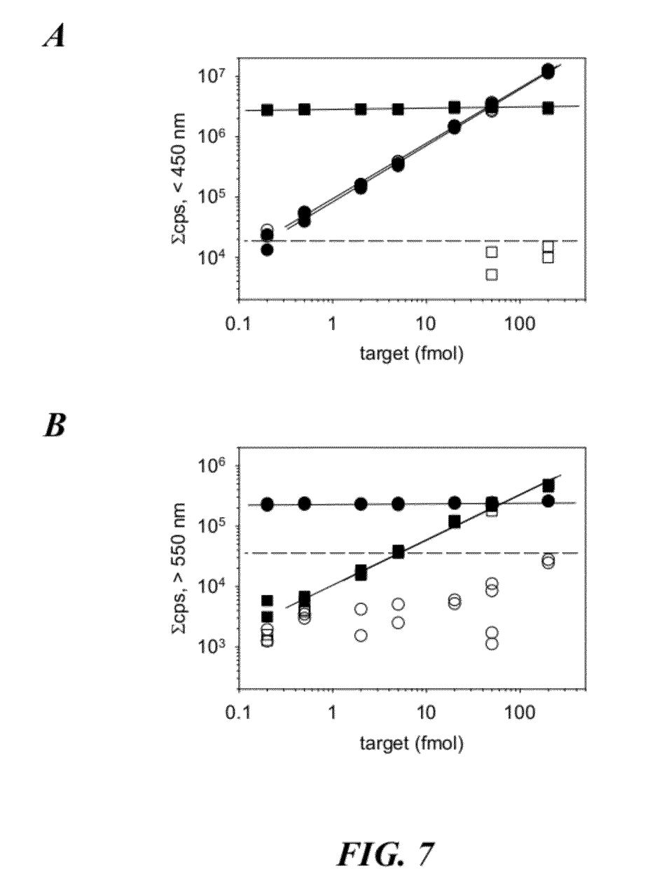

FIG. 7 demonstrates spectral-temporal resolved emissions from one example of several target dilutions. The deconvoluted response from EcoB1932-1947(−)HICS18 (corresponding to the first 12 s of emission measured at ≦450 nm, FIG. 7A) was linearly quantitative from 500 amol to 200 fmol of bacterial nucleic acid EcoB1921-1958(+) in the absence or presence of a high concentration (50 fmol) of fungal CalA1174-1217(+). EcoB1921-1958(+) curves in the absence or presence of CalA1174-1217 (+) are nearly indistinguishable except at the lowest target concentrations. As evidenced by the near zero slope (m<0.02) of the line linking emissions from solutions containing 50 fmol of bacterial target and various amounts of fungal target, the presence of fungal target (200 amol to 200 fmol) did not appreciably influence the emission responses of EcoB1932-1947(−)HICS18 from bacterial target.

The deconvoluted response from CalA1185-1206(−)HICS87 (corresponding to the emission measured at ≧550 nm between 12 and 184 s, FIG. 7B) was also linearly quantitative from about 500 amol to 200 fmol of fungal target in the absence or presence of a high concentration (50 fmol) of bacterial target. CalA1174-1217(+) curves in the absence or presence of EcoB1921-1958(+) are nearly indistinguishable except at the lowest target concentrations. Very high concentrations (e.g., 200 fmol) of bacterial target in the absence of fungal target yielded substantial long wavelength and long-lived emissions, effectively limiting the quantitative detection range of CalA1185-1206(−)HICS87 in the presence of possible bacterial target to just over 5 fmol to 200 fmol. Similar to EcoB1932-1947(−) HICS18, the emission responses from CalA1185-1206(−)HICS87 plus 50 fmol fungal target were minimally influenced by the presence of 200 amol to 200 fmol bacterial target (m<0.02). The slopes and intercepts of the specific target dilutions were similar to each other in the absence or presence of the other target, and repeated experiments demonstrated similar results.

From a single analyte point of view, the current results are close to or exceed the sensitivity reported in many literature examples. For example, in a heterogeneous, single-analyte, sandwich-type antibody assay with eight complex 2,7-dialkoxyAE labels (similar relative quantum yields as 2,7-dimethoxyAE) per secondary antibody, as little as 28 pmol of theophylline could be detected. (Natrajan et al., Anal. Biochem. 2010, 406:204-13). In a homogeneous, single-analyte molecular assay with a single C-linked AE label per linear DNA probe, 30 amol of C. trachomatis target could be detected. (Arnold et al., Clin. Chem. 1989, 35:1588-94). Finally, in another molecular assay in which single analytes were captured on magnetic particles in a ternary complex conjugated to horseradish peroxidase, chemiluminescent detection of DNA target was down to 10 amol. (Cai et al., Anal. Chem. 2010, 82:7178-84). However, compared with such tests, the present method has the major advantage of being suitable for simultaneous measurement of two analytes.

Other label and detection strategies have been used to quantify multiple analytes in individual, non-amplified samples. For example, Adamczyk et al. captured bovine serum albumin and myoglobin from a common solution, removed unbound materials by washing, and detected 100 fmol to 100 pmol of the analytes in the same solution by sequentially triggering aequorin label with Ca²⁺, recording luminescence, then triggering an AE label with alkaline peroxide and recording luminescence. (Adamczyk et al., Bioorg. Med. Chem. Lett. 2002, 12:395-98). Using acridinium- and benz[b]acridinium-labeled antibodies to follicle-stimulating hormone (FSH) and luteinizing hormone (LH), respectively, in a heterogeneous assay, Law et al. disclosed dual, wavelength-resolved detection of FSH and LH down to about 10-20 mIU/mL over about 10- and 5-fold concentration ranges. (U.S. Pat. No. 5,879,894). Nelson et al. used pairs of linear nucleic acid probes labeled with N-methyl AE that differed in the pKa of their phenolic leaving groups and, hence, in their emission time constants to homogeneously detect and quantify by time resolution as little as 500 amol of N. gonorrhoeae target or 150 amol of C. trachomatis target over a 20-fold concentration range in the absence or presence of the other analyte. (Nelson et al., Biochemistry 1996, 35:8429-38). While this last example demonstrated higher sensitivity for one of a pair of dual analytes, the dynamic range for the other analyte was narrower than in the current study (e.g., FIG. 7).

In summary, a new chemiluminescent label incorporating a 2,7-dimethoxy-AE unit has been synthesized and used to generate HICS probes. Emission constants of these probes have been characterized and compared to an existing HICS probe label design. Unimodal rise-and-decay of spectrograms over time from both probe types supports that emissions are from the generation of excited acridone species from AE- and from 2,7-dimethoxyAE-labeled probes. Peak emissions from 2,7-dimethoxyAE-labeled probes shift bathochromatically by about 58 nm from those of AE-labeled probes. Time-resolved chemiluminescent emissions indicate that 2,7-dimethoxyAE-labeled probes have emitter formation rates that are similar to those from AE-labeled probes but that they have substantially slower emission decay rates because of slower dark reactions of the AE. These differences in emission wavelengths and time courses advantageously support simultaneous spectral-temporal separation of signals from two probes in a dual wavelength luminometer. This allowed demonstration of a rapid (about 60 min/sample), sensitive (about 500 amol to 200 fmol), and homogeneous detection and quantification (dynamic range spanning up to 3 log concentration units) process without enzymatic amplification of two microbial target nucleic acids representing those that may be present in single environmental samples. As noted above, the method lends itself to providing further assay value by addition of extra detection channels to the luminometer and by including one or more additional spectrally and temporally resolved probes to other analytes or internal controls.

The above examples are included for illustrative purposes only and are not intended to limit the scope of the invention. Many variations to those described above are possible. Since modifications and variations to the examples described above will be apparent to those of skill in this art, it is intended that this invention be limited only by the scope of the appended claims.

1. A homogeneous assay for quantifying at least two target nucleic acid sequences in a sample, said assay comprising the steps of:
(a) sequentially or simultaneously:
(i) contacting the sample with a first hybridization induced chemiluminescent signal (HICS) probe comprising a first chemiluminescent molecule, under conditions allowing hybridization of the first probe to a first target nucleic acid sequence;
(ii) contacting the sample with a second HICS probe comprising a second chemiluminescent molecule, under conditions allowing hybridization of the second probe to a second target nucleic acid sequence that is different from the first target sequence;
(b) chemically triggering the first and second chemiluminescent molecules;
(c) detecting chemiluminescence emissions from the first and second chemiluminescent molecules, wherein the first and second chemiluminescent molecules have sufficiently different emission profiles to allow spectral and/or temporal resolution of the chemiluminescence emissions; and
(d) quantifying the target nucleic acid sequences in the sample based on the chemiluminescence emissions from the first and second chemiluminescent molecules.

2. The assay of claim 1, wherein the emission maxima of the first and second chemiluminescent molecules are separated by at least 50 nm.

3. The assay of claim 1, wherein the first and second chemiluminescent molecules comprise different acridinium ester (AE) moieties conjugated through an acridinium position other than C9 to a stem-loop oligonucleotide.

4. The assay of claim 3, wherein the first chemiluminescent molecule comprises an unsubstituted AE moiety, and the second chemiluminescent molecule comprises a 2,7-substituted AE moiety.

5. The assay of claim 3, wherein the AE moieties are conjugated through the N10 acridinium position to a stem-loop oligonucleotide.

6. The assay of claim 5, wherein the first chemiluminescent molecule comprises an unsubstituted AE moiety, and the second chemiluminescent molecule comprises a 2,7-substituted AE moiety.

7. The assay of claim 6, wherein the unsubstituted AE moiety is a 9-(2,6-dibromophenoxycarbonyl)-10-(3-carbonylpropyl)acridinium salt.

8. The assay of claim 6, wherein the substituted AE moiety is a 2,7-dimethoxy-9-(2,6-dibromophenoxycarbonyl)-10-(3-carbonylpropyl)acridinium salt.

9. The assay of claim 1, wherein the target nucleic acid sequences are selected from the group consisting of a pan-bacterial target sequence, a pan-fungal target sequence, a Chlamydia trachomatis (C. trachomatis) target sequence and a Neisseria gonorrhoeae (N. gonorrhoeae) target sequence.

10. The assay of claim 9, wherein the pan-bacterial target sequence comprises an Escherichia coli (E. coli) 23S rRNA fragment consisting of SEQ ID NO:1, allowing for a DNA equivalent thereof.

11. The assay of claim 10, wherein one of the probes comprises a stem-loop oligonucleotide having a target complementary base sequence consisting of SEQ ID NO:5, allowing for a DNA equivalent thereof.

12. The assay of claim 11, wherein the stem-loop oligonucleotide comprises a base sequence consisting of SEQ ID NO:9, SEQ ID NO:10 or SEQ ID NO:11, allowing for DNA equivalents thereof.

13. The assay of claim 9, wherein the pan-fungal target sequence comprises a Candida albicans (C. albicans) 18S rRNA fragment consisting of SEQ ID NO:2, allowing for a DNA equivalent thereof.

14. The assay of claim 13, wherein the one of the probes comprises a stem-loop oligonucleotide having a target complementary sequence consisting of SEQ ID NO:6, allowing for an RNA equivalent thereof.

15. The assay of claim 14, wherein the stem-loop oligonucleotide comprises a base sequence consisting of SEQ ID NO:12 or SEQ ID NO:13, allowing for RNA equivalents thereof.

16. The assay of claim 9, wherein the C. trachomatis target sequence comprises a 23S rRNA fragment consisting of SEQ ID NO:3, allowing for a DNA equivalent thereof.

17. The assay of claim 16, wherein one of the probes comprises a stem-loop oligonucleotide having a target complementary sequence consisting of SEQ ID NO:7, allowing for a DNA equivalent thereof.

18. The assay of claim 17, wherein the stem-loop oligonucleotide comprises a base sequence consisting of SEQ ID NO:14, allowing for a DNA equivalent thereof.

19. The assay of claim 9, wherein the N. gonorrhoeae target sequence comprises a 16S rRNA fragment consisting of SEQ ID NO:4, allowing for a DNA equivalent thereof.

20. The assay of claim 19, wherein one of the probes comprises a stem-loop oligonucleotide having a target complementary base sequence consisting of SEQ ID NO:8, allowing for a DNA equivalent thereof.

21. The assay of claim 20, wherein the stem-loop oligonucleotide comprises a base sequence consisting of SEQ ID NO:15, allowing for a DNA equivalent thereof.

22. A composition comprising a 2,7-dimethoxy-9-(2,6-dibromophenoxycarbonyl)-10-(3-carbonylpropyl)acridinium salt.

23. The composition of claim 22, wherein the salt is 2,7-dimethoxy-9-(2,6-dibromophenoxycarbonyl)-10-(3-carbonylpropyl)acridinium iodide.

24. The composition of claim 22, wherein the 2,7-dimethoxy-9-(2,6-dibromophenoxycarbonyl)-10-(3-carbonylpropyl)acridinium salt is directly or indirectly conjugated to a small organic biomolecule, a macromolecule, a viral particle, a sub-cellular component, or a cell.

25. The composition of claim 24, wherein the macromolecule is selected from the group consisting of a protein, a peptide, a nucleic acid, an oligonucleotide, a polysaccharide, an oligosaccharide, a glycoprotein, a glycosaminoglycan, a lectin, a lipoprotein, a lipopolysaccharide, a hormone, a toxin, a cytokine, and a combination thereof.

26. The composition of claim 25, wherein the oligonucleotide is a stem-loop oligonucleotide.

27. A kit comprising the composition of claim 22 and reagent means for triggering a chemiluminescence emission.

28. The kit for detecting and/or quantifying a target nucleic acid sequence in a sample, the kit comprising the composition of claim 26 and reagent means for triggering a chemiluminescence emission.

29. A kit for detecting and/or quantifying at least two target nucleic acid sequences in a sample, the kit comprising the probes according to claim 6 and reagent means triggering a chemiluminescence emission.

30. The kit of claim 29, wherein the unsubstituted AE moiety is a 9-(2,6-dibromophenoxycarbonyl)-10-(3-carbonylpropyl)acridinium salt.

31. The kit of claim 29, wherein the substituted AE moiety is a 2,7-dimethoxy-9-(2,6-dibromophenoxycarbonyl)-10-(3-carbonylpropyl)acridinium salt.
